SEQUENCE LISTING (10 sequences)

SEQ ID NO: 1 - DNA, 20 bp, Artificial Sequence
  Synthetic primer
cacacatccagagctctatg

SEQ ID NO: 2 - DNA, 54 bp, Artificial Sequence
  Synthetic primer
atactatattaccctgttatccctagcgtaactacaaatacaagttttaagctg

SEQ ID NO: 3 - DNA, 56 bp, Artificial Sequence
  Synthetic primer
tatataagttacgctagggataacagggtaatataggctgtgtatgtgttagggtg

SEQ ID NO: 4 - DNA, 20 bp, Artificial Sequence
  Synthetic primer
ggtgaccttcaggtgcacca

SEQ ID NO: 5 - DNA, 2071 bp, Artificial Sequence
  PCR product
cacacatccagagctctatgaagagatgggtatcaaaccccccaagggagtcattctttacggcgctcccggaaccggaaagacactgctggccaaggccgtcgccaaccagacttctgctacatttttgcgaatcgttggatccgagctgatccaaaagtacttgggagacggtccccgactgtgtcgacagattttccagattgccgcagaacacgcaccgtccattgttttcatcgatgagattgacgctattggaaccaaacgatacgagtcgacgtctggcggagagcgagaggtgcagcggaccatgctggagcttctgaaccagctggatggattcgacgaccgaggagacgtcaaggtaattatggctacaaacaagattgagtcgctggaccctgcactgattcgacctggtcgaatcgaccgaaagattctgtttgagaatcccgactccaccaccaagcgaaagattatgggcatccacacctccaagatgaacctcaacgacgatgttgatctcgaggagtttgtgtcttccaaggatgaactttctggagccgacatcaaggccatgtgcaccgaggcaggtctgcttgctctgcgagagagacgaatgcgggtcactgctgaggatttccgaaccgctaaagagcgggtcatgaagaacaaggtggaggagaaccttgagggtctctacctgtaagcatagaagcattaacgtatgactataataaatacatcaatgaggtggaggttgacggggacttcttcgtatacatactaacatctaacatctacagtagatcgaataacttgcaacggtacatgccgtcatagaaagacgtttggtagaaaatgcctaatagagacaacacacattccaatacacagacagttacaaccagcagccacattgttatataaatgtttaccaagtgagcctaaccagtgggtgtaagtcatacatcatcattcattatttagtcatgatccatgaaatgtttatgcaatacgtttcacagttcattgggtcagcttaaaacttgtatttgttatataagttacgctagggataacagggtaatatagtatatagctgtgtatgtgttagggtgggttttggtgtgttggaaaggagtgccagaaaagagatcggtctagctttatcctctggcctgcggctcaccccagatttgaactttgggtttgtcgggctagtctcggggcgcgtgtgggggagatgcatcaataaccgcataatagtggtcccactgcaggtgtttggaggtggagctgaaacttttatttcctaatatcgtattcaagctgtctccaaaggagcattcatcctcgcaactcctcggactagctccaaaaggcatttttagccgcgtctgcgtacaggtaagggatcccccccacattatccacccagcattagtcttgcgttatgtacactcaacagagttcgtcgattgccgccgaagcggacatcttcgttgggtgagggaccaggcttgtggtatattaattggagaggagggccgagagggttggcgagaagttacaaattgaactaggagtacacatagaatgatataccttctacttctactgacggtcgtagtcacaatacgtgcgtgtgcacaatacgtgcgtgtgtacatctgagttataacttgcactgggttgtcagtgatgaacccacagtgtgattatgtcctgcccagtgtacaagtacagcactattatgggtgttgcaccctatccatctatatacccaccatgttagccccgtaagtgagattgattcccttcatgcgctccacaagtacagtacatagtcatcggcatgcatattatatacctacagcatgtacagtatgtactgtaatagtgggagatcctacagtatttgtagtgctttcttggtggacatgcatactttgcaccagtgtcgaaaatatcgtacttgtacttaaccactttttgtacttttataacgtgcgtcaaagcctcttaattgatgacgctccagctattattggtgcacctgaaggtcacc

SEQ ID NO: 6 - DNA, 5590 bp, Artificial Sequence
  Vector pCRBluntIITopo::P_T_3HIBDH_Yl
cacacatccagagctctatgaagagatgggtatcaaaccccccaagggagtcattctttacggcgctcccggaaccggaaagacactgctggccaaggccgtcgccaaccagacttctgctacatttttgcgaatcgttggatccgagctgatccaaaagtacttgggagacggtccccgactgtgtcgacagattttccagattgccgcagaacacgcaccgtccattgttttcatcgatgagattgacgctattggaaccaaacgatacgagtcgacgtctggcggagagcgagaggtgcagcggaccatgctggagcttctgaaccagctggatggattcgacgaccgaggagacgtcaaggtaattatggctacaaacaagattgagtcgctggaccctgcactgattcgacctggtcgaatcgaccgaaagattctgtttgagaatcccgactccaccaccaagcgaaagattatgggcatccacacctccaagatgaacctcaacgacgatgttgatctcgaggagtttgtgtcttccaaggatgaactttctggagccgacatcaaggccatgtgcaccgaggcaggtctgcttgctctgcgagagagacgaatgcgggtcactgctgaggatttccgaaccgctaaagagcgggtcatgaagaacaaggtggaggagaaccttgagggtctctacctgtaagcatagaagcattaacgtatgactataataaatacatcaatgaggtggaggttgacggggacttcttcgtatacatactaacatctaacatctacagtagatcgaataacttgcaacggtacatgccgtcatagaaagacgtttggtagaaaatgcctaatagagacaacacacattccaatacacagacagttacaaccagcagccacattgttatataaatgtttaccaagtgagcctaaccagtgggtgtaagtcatacatcatcattcattatttagtcatgatccatgaaatgtttatgcaatacgtttcacagttcattgggtcagcttaaaacttgtatttgttatataagttacgctagggataacagggtaatatagtatatagctgtgtatgtgttagggtgggttttggtgtgttggaaaggagtgccagaaaagagatcggtctagctttatcctctggcctgcggctcaccccagatttgaactttgggtttgtcgggctagtctcggggcgcgtgtgggggagatgcatcaataaccgcataatagtggtcccactgcaggtgtttggaggtggagctgaaacttttatttcctaatatcgtattcaagctgtctccaaaggagcattcatcctcgcaactcctcggactagctccaaaaggcatttttagccgcgtctgcgtacaggtaagggatcccccccacattatccacccagcattagtcttgcgttatgtacactcaacagagttcgtcgattgccgccgaagcggacatcttcgttgggtgagggaccaggcttgtggtatattaattggagaggagggccgagagggttggcgagaagttacaaattgaactaggagtacacatagaatgatataccttctacttctactgacggtcgtagtcacaatacgtgcgtgtgcacaatacgtgcgtgtgtacatctgagttataacttgcactgggttgtcagtgatgaacccacagtgtgattatgtcctgcccagtgtacaagtacagcactattatgggtgttgcaccctatccatctatatacccaccatgttagccccgtaagtgagattgattcccttcatgcgctccacaagtacagtacatagtcatcggcatgcatattatatacctacagcatgtacagtatgtactgtaatagtgggagatcctacagtatttgtagtgctttcttggtggacatgcatactttgcaccagtgtcgaaaatatcgtacttgtacttaaccactttttgtacttttataacgtgcgtcaaagcctcttaattgatgacgctccagctattattggtgcacctgaaggtcaccagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccctta

SEQ ID NO: 7 - DNA, 6899 bp, Artificial Sequence
  pCRBluntIITopo::P_T_3HIBDH_Yl_ura
cacacatccagagctctatgaagagatgggtatcaaaccccccaagggagtcattctttacggcgctcccggaaccggaaagacactgctggccaaggccgtcgccaaccagacttctgctacatttttgcgaatcgttggatccgagctgatccaaaagtacttgggagacggtccccgactgtgtcgacagattttccagattgccgcagaacacgcaccgtccattgttttcatcgatgagattgacgctattggaaccaaacgatacgagtcgacgtctggcggagagcgagaggtgcagcggaccatgctggagcttctgaaccagctggatggattcgacgaccgaggagacgtcaaggtaattatggctacaaacaagattgagtcgctggaccctgcactgattcgacctggtcgaatcgaccgaaagattctgtttgagaatcccgactccaccaccaagcgaaagattatgggcatccacacctccaagatgaacctcaacgacgatgttgatctcgaggagtttgtgtcttccaaggatgaactttctggagccgacatcaaggccatgtgcaccgaggcaggtctgcttgctctgcgagagagacgaatgcgggtcactgctgaggatttccgaaccgctaaagagcgggtcatgaagaacaaggtggaggagaaccttgagggtctctacctgtaagcatagaagcattaacgtatgactataataaatacatcaatgaggtggaggttgacggggacttcttcgtatacatactaacatctaacatctacagtagatcgaataacttgcaacggtacatgccgtcatagaaagacgtttggtagaaaatgcctaatagagacaacacacattccaatacacagacagttacaaccagcagccacattgttatataaatgtttaccaagtgagcctaaccagtgggtgtaagtcatacatcatcattcattatttagtcatgatccatgaaatgtttatgcaatacgtttcacagttcattgggtcagcttaaaacttgtatttgttatataagttacgctagggataacagggtaatcataacttcgtatagcatacattatacgaagttattctgaattccgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgctgttcagcttgtatgatggtcagacgacctgtctgatcgagtatgtatggtactgcacaacctgtgtatccgcatggtctgtccaatggggcatgttgttgtgtttctcggaattcgtataacttcgtatagcaggagttatccgaagcgatatagggataacagggtaatatagtatatagctgtgtatgtgttagggtgggttttggtgtgttggaaaggagtgccagaaaagagatcggtctagctttatcctctggcctgcggctcaccccagatttgaactttgggtttgtcgggctagtctcggggcgcgtgtgggggagatgcatcaataaccgcataatagtggtcccactgcaggtgtttggaggtggagctgaaacttttatttcctaatatcgtattcaagctgtctccaaaggagcattcatcctcgcaactcctcggactagctccaaaaggcatttttagccgcgtctgcgtacaggtaagggatcccccccacattatccacccagcattagtcttgcgttatgtacactcaacagagttcgtcgattgccgccgaagcggacatcttcgttgggtgagggaccaggcttgtggtatattaattggagaggagggccgagagggttggcgagaagttacaaattgaactaggagtacacatagaatgatataccttctacttctactgacggtcgtagtcacaatacgtgcgtgtgcacaatacgtgcgtgtgtacatctgagttataacttgcactgggttgtcagtgatgaacccacagtgtgattatgtcctgcccagtgtacaagtacagcactattatgggtgttgcaccctatccatctatatacccaccatgttagccccgtaagtgagattgattcccttcatgcgctccacaagtacagtacatagtcatcggcatgcatattatatacctacagcatgtacagtatgtactgtaatagtgggagatcctacagtatttgtagtgctttcttggtggacatgcatactttgcaccagtgtcgaaaatatcgtacttgtacttaaccactttttgtacttttataacgtgcgtcaaagcctcttaattgatgacgctccagctattattggtgcacctgaaggtcaccagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccctta

SEQ ID NO: 8 - DNA, 3380 bp, Artificial Sequence
  PCR product II
cacacatccagagctctatgaagagatgggtatcaaaccccccaagggagtcattctttacggcgctcccggaaccggaaagacactgctggccaaggccgtcgccaaccagacttctgctacatttttgcgaatcgttggatccgagctgatccaaaagtacttgggagacggtccccgactgtgtcgacagattttccagattgccgcagaacacgcaccgtccattgttttcatcgatgagattgacgctattggaaccaaacgatacgagtcgacgtctggcggagagcgagaggtgcagcggaccatgctggagcttctgaaccagctggatggattcgacgaccgaggagacgtcaaggtaattatggctacaaacaagattgagtcgctggaccctgcactgattcgacctggtcgaatcgaccgaaagattctgtttgagaatcccgactccaccaccaagcgaaagattatgggcatccacacctccaagatgaacctcaacgacgatgttgatctcgaggagtttgtgtcttccaaggatgaactttctggagccgacatcaaggccatgtgcaccgaggcaggtctgcttgctctgcgagagagacgaatgcgggtcactgctgaggatttccgaaccgctaaagagcgggtcatgaagaacaaggtggaggagaaccttgagggtctctacctgtaagcatagaagcattaacgtatgactataataaatacatcaatgaggtggaggttgacggggacttcttcgtatacatactaacatctaacatctacagtagatcgaataacttgcaacggtacatgccgtcatagaaagacgtttggtagaaaatgcctaatagagacaacacacattccaatacacagacagttacaaccagcagccacattgttatataaatgtttaccaagtgagcctaaccagtgggtgtaagtcatacatcatcattcattatttagtcatgatccatgaaatgtttatgcaatacgtttcacagttcattgggtcagcttaaaacttgtatttgttatataagttacgctagggataacagggtaatcataacttcgtatagcatacattatacgaagttattctgaattccgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgctgttcagcttgtatgatggtcagacgacctgtctgatcgagtatgtatggtactgcacaacctgtgtatccgcatggtctgtccaatggggcatgttgttgtgtttctcggaattcgtataacttcgtatagcaggagttatccgaagcgatatagggataacagggtaatatagtatatagctgtgtatgtgttagggtgggttttggtgtgttggaaaggagtgccagaaaagagatcggtctagctttatcctctggcctgcggctcaccccagatttgaactttgggtttgtcgggctagtctcggggcgcgtgtgggggagatgcatcaataaccgcataatagtggtcccactgcaggtgtttggaggtggagctgaaacttttatttcctaatatcgtattcaagctgtctccaaaggagcattcatcctcgcaactcctcggactagctccaaaaggcatttttagccgcgtctgcgtacaggtaagggatcccccccacattatccacccagcattagtcttgcgttatgtacactcaacagagttcgtcgattgccgccgaagcggacatcttcgttgggtgagggaccaggcttgtggtatattaattggagaggagggccgagagggttggcgagaagttacaaattgaactaggagtacacatagaatgatataccttctacttctactgacggtcgtagtcacaatacgtgcgtgtgcacaatacgtgcgtgtgtacatctgagttataacttgcactgggttgtcagtgatgaacccacagtgtgattatgtcctgcccagtgtacaagtacagcactattatgggtgttgcaccctatccatctatatacccaccatgttagccccgtaagtgagattgattcccttcatgcgctccacaagtacagtacatagtcatcggcatgcatattatatacctacagcatgtacagtatgtactgtaatagtgggagatcctacagtatttgtagtgctttcttggtggacatgcatactttgcaccagtgtcgaaaatatcgtacttgtacttaaccactttttgtacttttataacgtgcgtcaaagcctcttaattgatgacgctccagctattattggtgcacctgaaggtcacc

SEQ ID NO: 9 - DNA, 20 bp, Artificial Sequence
  Synthetic primer
gagtcgcagattcaggaaat

SEQ ID NO: 10 - DNA, 20 bp, Artificial Sequence
  Synthetic primer
tcaccttctgatcacggtgt

(19) United States
(12) Patent Application Publication
(10) Pub. No.: US 2015/0218600 A1
(43) Pub. Date: Aug. 6, 2015

(54) BIOTECHNOLOGICAL PREPARATION OF 3-HYDROXYISOBUTYRIC ACID

(71) Applicants: Thomas Haas, Muenster (DE); Steffen Schaffer, Herten (DE); Markus Poetter, Shanghai (CN); Mirja Wessel, Bochum (DE); Jan Christoph Pfeffer, Hanau (DE); Christian Gehring, Marl (DE); Nicole Kirchner, Recklinghausen (DE); Eva Maria Wittmann, Traunreut (DE)

(72) Inventors: Thomas Haas, Muenster (DE); Steffen Schaffer, Herten (DE); Markus Poetter, Shanghai (CN); Mirja Wessel, Bochum (DE); Jan Christoph Pfeffer, Hanau (DE); Christian Gehring, Marl (DE); Nicole Kirchner, Recklinghausen (DE); Eva Maria Wittmann, Traunreut (DE)

(73) Assignee: Evonik Degussa GmbH, Essen (DE)

(21) Appl. No.: 14/363,178

(22) PCT Filed: Nov. 14, 2012
(86) PCT No.: PCT/EP2012/072594

§ 371 Date: Jun. 5, 2014

(30) Foreign Application Priority Data
Dec. 5, 2011 (EP) 11191923.9

(51) Int. Cl. C12P 7/42; C12N 9/88; C12N 9/12; C12N 9/02; C12N 9/00; C12N 9/10

(52) U.S. Cl. CPC C12P 7/42; C12N 9/93; C12N 9/13; C12N 9/1217; C12N 9/001; C12N 9/88; C12Y 103/99002; C12Y 207/02007; C12Y 602/01002; C12Y 402/01017

(57) ABSTRACT

The invention relates to a method comprising the steps a) providing isobutyric acid, b) bringing isobutyric acid into contact with the combination of isobutyrate kinase and phosphotransisobutyrylase and/or isobutyryl-coenzyme A synthetase/ligase and/or isobutyrate-coenzyme A transferase, c) bringing the product from step a) into contact with isobutyryl-coenzyme A dehydrogenase, d) bringing the product from step b) into contact with methacrylyl-coenzyme A hydratase, and e) hydrolyzing the product from step d) to form 3-hydroxyisobutyric acid, where at least one of the enzymes is used in the form of a cell which, compared to its wildtype, comprises a reduced activity of a 3-hydroxyisobutyric acid dehydrogenase or a variant thereof, a cell which has at least one enzyme from the group comprising isobutyryl-coenzyme A synthetase/ligase, isobutyrate-coenzyme A transferase, isobutyrate kinase, phosphotransisobutyrylase, isobutyryl-coenzyme A dehydrogenase, methacrylyl-coenzyme A hydratase and 3-hydroxyisobutyryl-coenzyme A hydrolase and, compared to its wildtype, a reduced activity of a 3-hydroxyisobutyric acid dehydrogenase or a variant thereof, wherein the cell preferably has, in addition, a monooxygenase, more preferably a monooxygenase of the alkBGT type or a variant thereof and the use of such a cell for preparing 3-hydroxyisobutyric acid.

The invention relates to a method comprising the steps a) providing isobutyric acid, b) bringing isobutyric acid into contact with the combination of isobutyrate kinase and phosphotransisobutyrylase and/or isobutyryl-coenzyme A synthetase/ligase and/or isobutyrate-coenzyme A transferase, c) bringing the product from step b) into contact with isobutyryl-coenzyme A dehydrogenase, d) bringing the product from step c) into contact with methacrylyl-coenzyme A hydratase, and e) hydrolyzing of the product from step d) to form 3-hydroxyisobutyric acid, where at least one of the enzymes is provided in the form of a cell, which, compared to its wildtype, comprises a reduced activity of a 3-hydroxyisobutyric acid dehydrogenase or a variant thereof, a cell which has at least one enzyme from the group comprising isobutyryl-coenzyme A synthetase/ligase, isobutyrate-coenzyme A transferase, isobutyrate kinase, phosphotransisobutyrylase, isobutyryl-coenzyme A dehydrogenase, methacrylyl-coenzyme A hydratase and 3-hydroxyisobutyryl-coenzyme A hydrolase and, compared to its wildtype, a reduced activity of a 3-hydroxyisobutyric acid dehydrogenase or a variant thereof, wherein the cell preferably has, in addition, a monooxygenase, more preferably an alkane hydroxylase of the alkBGT type or a variant thereof and the use of such a cell for preparing 3-hydroxyisobutyric acid.

Methacrylic acid constitutes one of the most important industrially produced chemicals. In the form of its monomeric methyl ester, it is required as a polymerisation reactant for preparing polymethyl methacrylate, being known to the public under the trade name Plexiglas, and is indispensible in many areas of application. Examples of the use of polymethacrylate include dentistry, where it is used for prostheses, the automotive industry, in which it is used as the glass for indicator and tail lights, optics, in particular as a material for contact lenses and spectacle glass, the building trade, where it is used as polymer concrete and also as a two-component adhesive, the textile industry as a constituent of polyacrylic fibres, and in the domestic home as a material for items such as dishes and cutlery.

Methacrylic acid is conventionally prepared starting from fossil raw materials such as oil. For example, isobutylene and tertiary butanol can be converted to methacrolein, which is then further oxidised to methacrylate (William Bauer, Jr. “Methacrylic Acid and Derivatives” in Ullmann's Encyclopedia of Industrial Chemistry 2002, Wiley-VCH, Weinheim). Alternatively, amidosulphates of methacrylic acid, which are produced starting from the corresponding 2-hydroxynitriles, can be hydrolysed to form methacrylic acid. The industrial production of methacrylic acid using such methods depends however not only on a continuous supply of fossil reactants, but also takes place with the consumption of considerable quantities of aggressive, environmentally harmful chemicals. For example, the production of 1 kg of methacrylic acid by hydrolysis of amidosulphates of methacrylic acid requires 1.6 kg of sulphuric acid.

In order to overcome the dependence on fossil raw materials as a source of energy and reactants for industrial syntheses, diverse efforts are currently being adopted, which are aimed at producing industrially required fine chemicals biotechnologically on the basis of renewable raw materials. In the case of methacrylic acid and the respective methyl ester, a biotechnological synthesis route via 3-hydroxyisobutyric acid, which can be readily dehydrated chemically or enzymatically to form methacrylic acid, is suitable (William Bauer, Jr. “Methacrylic Acid and Derivatives” in Ullmann's Encyclopedia of Industrial Chemistry 2002, Wiley-VCH, Weinheim). The prior art teaches the preparation of 3-hydroxyisobutyric acid from isobutyric acid using wildtype isolates of bacteria and yeasts (Hasegawa et al., 1981; Hasegawa et al., 1982, WO 2007/141208 A2 and WO 2008/119738 A1). Isobutyric acid is used exclusively as the substrate for preparing 3-hydroxyisobutyric acid by suitable strains, for example by Candida rugosa. The abovementioned patent applications, WO 2007/141208 A2 and WO 2008/119738 A1, describe a genetically modified cell and method for preparing 3-hydroxyisobutyric acid from carbohydrates, glycerol, carbon dioxide, methanol, L-valine, L-glutamate, CO, synthesis gas, methane etc. and also its further chemical conversion to methacrylic acid or methacrylic acid esters.

The preparation of 3-hydroxyisobutyric acid via the biotechnological methods described above is currently however uneconomical. A significant drawback here is the high raw material costs for isobutyric acid and, concomitantly, at times the low yield during their conversion or use of appropriate microorganisms.

Against this background, it is the object of the present invention to develop an improved method for the biotechnological preparation of 3-hydroxyisobutyric acid, which is superior to the methods described in the prior art with regard to yield, purity and the resources required.

Furthermore, the object of the present invention consists in developing a biotechnological method for preparing 3-hydroxyisobutyric acid starting from unsubstituted, particularly non-heteroatom-containing, alkanes.

Furthermore, the object of the present invention is to develop a biotechnological method for obtaining 3-hydroxyisobutyric acid starting from renewable raw materials and/or without the use of, or with less use of, harmful reactants, intermediates, catalysts or by-products.

These and other objects are achieved by the subject matter of the present application and particularly also by the subject matter of the accompanying independent claims, with embodiments arising from the dependent claims.

The object is achieved according to the invention in a first aspect by a method comprising the following steps:
a) providing isobutyric acid,
b) bringing isobutyric acid into contact with
   the combination of isobutyrate kinase and phosphotransisobutyrylase and/or
   isobutyryl-coenzyme A synthetase/ligase and/or
   isobutyrate-coenzyme A transferase,
c) bringing the product from step b) into contact with isobutyryl-coenzyme A dehydrogenase,
d) bringing the product from step c) into contact with methacrylyl-coenzyme A hydratase, and
e) hydrolyzing the product from step d) to form 3-hydroxyisobutyric acid,
wherein at least one of the enzymes used in the steps b), c) and d) from the group comprising isobutyrate kinase, phosphotransisobutyrylase, isobutyryl-coenzyme A synthetase/ligase and isobutyrate-coenzyme A transferase, preferably all, enzymes is used in the form of a cell, which, compared to its wildtype, has a reduced activity of a 3-hydroxyisobutyric acid dehydrogenase or a variant thereof.

In a first embodiment of the first aspect, the isobutyric acid is formed by bringing isobutane into contact with a monooxygenase, preferably an alkane hydroxylase, more preferably one of the alkBGT type or a variant thereof.

In a second embodiment of the first aspect, which also constitutes an embodiment of the first embodiment, the hydrolysis in step e) is achieved by bringing the product from step d) into contact with a 3-hydroxyisobutyryl-coenzyme A hydrolase.

In a third embodiment of the first aspect, which also constitutes an embodiment of the first and second embodiment, the cell has both the isobutyryl-coenzyme A dehydrogenase in step c) and the methacrylyl-coenzyme A hydratase in step d) and
the combination of isobutyrate kinase and phosphotransisobutyrylase and/or
isobutyryl-coenzyme A synthetase/ligase and/or
isobutyrate-coenzyme A transferase.

In a fourth embodiment of the first aspect, which also constitutes an embodiment of the first to third embodiment, the cell additionally has an alkane hydroxylase, preferably one of the alkBGT type or a variant thereof.

In a fifth embodiment of the first aspect, which also constitutes an embodiment of the first to fourth embodiment, the 3-hydroxyisobutyric acid dehydrogenase is XP_{—}504911.1 or a variant thereof.

The object in a second aspect is achieved, according to the invention, by a cell which has at least one enzyme from the group comprising isobutyryl-coenzyme A synthetase/ligase, isobutyrate-coenzyme A transferase, isobutyrate kinase, phosphotransisobutyrylase, isobutyryl-coenzyme A dehydrogenase, methacrylyl-coenzyme A hydratase and 3-hydroxyisobutyryl-coenzyme A hydrolase and, compared to its wildtype, a reduced activity of a 3-hydroxyisobutyric acid dehydrogenase or a variant thereof.

In a first embodiment of the second aspect the cell has, in addition to an isobutyryl-coenzyme A dehydrogenase, and in addition to a methacrylyl-coenzyme A hydratase,
the combination of isobutyrate kinase and phosphotransisobutyrylase and/or
isobutyryl-coenzyme A synthetase/ligase and/or
isobutyrate-coenzyme A transferase,
preferably furthermore a 3-hydroxyisobutyryl-CoA hydrolase.

In a second embodiment of the second aspect, which also constitutes an embodiment of the first embodiment, the cell further comprises an alkane hydroxylase, preferably one of the alkBGT type or a variant thereof.

In a third embodiment of the second aspect, which also constitutes an embodiment of the first embodiment, the 3-hydroxyisobutyric acid dehydrogenase is Yali0F02607g (XP_{—}504911) or a variant thereof.

In a third aspect the object of the present invention is achieved by using the cell according to any of Claims 7 to 10 for preparing 3-hydroxyisobutyric acid.

In one embodiment of the third aspect, the 3-hydroxyisobutyric acid dehydrogenase is XP_{—}504911.1 or a variant thereof.

In a further embodiment of the first, second or third aspect, the cell is a bacterial or lower eukaryotic cell.

In a further embodiment of the first, second or third aspect, the cell is a yeast cell from the group of genera which comprises Yarrowia, Candida, Saccharomyces, Schizosaccharomyces and Pichia and is preferably Yarrowia lipolytica.

In a fourth aspect the object of the present invention is achieved by a reaction mixture comprising the cell according to the second aspect and also isobutane or isobutyric acid.

The inventors of the present invention have surprisingly established that the inactivation of a gene which codes for an enzyme identified as 3-hydroxyisobutyric acid dehydrogenase in a microorganism leads to an increased yield of 3-hydroxyisobutyric acid.

The inventors have further surprisingly found that it is possible to prepare 3-hydroxyisobutyric acid biotechnologically, starting from alkane reactants, particularly isobutane.

Conducting the method according to the invention initially requires the provision of isobutyric acid. Firstly, there is the option of using commercially available isobutyric acid. Secondly, there is the option to prepare isobutyric acid using isolated enzymes or whole organisms with suitable catalytic capability, starting from other reactants, for example by cultivation of an organism which naturally produces isobutyric acid. In a preferred embodiment, the isobutyric acid is prepared by bringing isobutane, or another suitable alkane precursor, into contact with a suitable monooxygenase, preferably alkane hydroxylase, which, in a particularly preferred embodiment, is an alkane hydroxylase of the alk-BGT type or a variant thereof. In a preferred embodiment, the term “alkane hydroxylase”, as used here, is understood to mean an oxidoreductase which has the ability to oxidise saturated hydrocarbons, particularly isobutane or 3-hydroxyisobutane, to give the carboxylic acid, preferably at a terminal carbon atom. The prior art describes a series of suitable microorganisms and enzymes. For example, Patel et al. (Journal of Applied Biochemistry, 1983, 5 (1-2), 107-120) describe 16 new bacterial strains which have the ability to oxidise gaseous alkanes with a chain length of from C2 to C4 to give the corresponding methyl ketones, secondary and primary alcohols and aldehydes. Grant et al. (2011) describe the oxidation of alkanes to corresponding acids by means of the alkB alkane hydroxylase (Grant, C, Woodley, J & Baganz, F 2011, ‘Whole-cell bio-oxidation of n-dodecane using the alkane hydroxylase system of P. putida GPo1 expressed in E. coli’, Enzyme and Microbial Technology, vol 48, no. 6-7, pp. 480-486).

On the other hand, there is the option to prepare isobutyric acid using suitable strains of microorganisms which, naturally or by genetic modification, are endowed with metabolic pathways which include producing isobutyric acid by feeding with suitable carbon sources, e.g. glucose. Examples of microorganisms include e.g. Yarrowia lipolytica, Candida rugosa, Hanseniaspora valbyensis, Hansenula anomala, Trichosporon aculeatum, Trichosporon fennicum, Endomyces reessii, Geotrichum loubieri, Micrococcus flavus, Micrococcus luteus, Micrococcus lysodeikticum, Candida parapsilosis, Pichia membranaefaciens, Torulopsis candida, Coccidioides posadasii, Coccidioides immitis, Verticillium dahliae, Gibberella zeae, Thielavia terrestris, Metarhizium acridum, Magnaporthe oryzae, Sordaria macrospora, Metarhizium nisopliae, Ajellomyces dermatitidis, Chaetomium globosum, Paracoccidioides brasiliensis, Nectria haematococca, Neurospora tetrasperma, Chaetomium thermophilum and Neurospora crassa.

In a preferred embodiment, the isobutyric acid is provided by oxidation of isobutane by the oxidoreductase alkB from the alkBGT system of Pseudomonas putida or a variant thereof. AlkB is an oxidoreductase from the alkBGT system of Pseudomonas putida, which is known for its alkane hydroxylase activity. This is dependent on two further polypeptides, alkG and alkT. AlkT is characterised as FAD-dependent rubredoxin reductase, which transfers electrons from NADH to alkG. AlkG is a rubredoxin, an iron containing redox protein, which functions as a direct electron donor for alkB. In a preferred embodiment, the term “alkane hydroxylase of the alkBGT type”, as used here, is understood to mean a membrane-bound alkane monooxidase. In a further preferred embodiment the same term “alkane hydroxylase of the alkBGT type” is understood to mean a polypeptide with a sequence homology of, with increasing preference, at least 75, 80, 85, 90, 92, 94, 96, 98 or 99% to the sequence of the alkB of Pseudomonas putida Gpo1 (data bank code: CAB54050.1). In a further preferred embodiment, the term is understood to mean a cytochrome-independent monooxygenase. In a further preferred embodiment, the term “alkane hydroxylase of the alkBGT type” is understood to mean a cytochrome-independent monooxygenase which uses a rubredoxin or homologue as electron donor. In a particularly preferred embodiment, the term is understood to mean a membrane-bound, cytochrome-independent alkane monooxygenase of, with increasing preference, at least 60, 70, 80, 80, 85, 90, 92, 94, 96, 98 or 99% to the sequence of the alkB of Pseudomonas putida Gpo1, which requires as electron donor at least alkG (CAB54052.1), but preferably the combination of alkG with the reductase alkT (CAB54063.1), wherein alkG and/or alkT may also be a homologue of the respective polypeptide. The term “sequence”, as used here, may refer to the amino acid sequence of a polypeptide and/or its nucleic acid coding sequence. In a further preferred embodiment, “an oxidoreductase of the alkB type”, as used here, is a cytochrome-independent oxidoreductase, i.e. an oxidoreductase which does not include cytochrome as cofactor.

There is the option, for this purpose, to bring purified components of the alkBGT system into contact with isobutane, particularly alkB. In a preferred embodiment, isobutane is brought into contact with an alkBGT-containing whole-cell catalyst; in a most preferred embodiment expressed with a recombinant E. coli strain of the heterologous alkBGT.

The teaching of the present invention may be implemented not only by using the exact amino acid or nucleic acid sequences of the biological macromolecules described herein, but also by using variants of such macromolecules, which may be obtained by deletion, addition or substitution of one, or more than one, amino acids or nucleic acids. In a preferred embodiment, the term “variant” of a nucleic acid sequence or an amino acid sequence, hereinbelow used synonymously and interchangeably with the term “homologue”, as used here, means another nucleic acid or amino acid sequence which has an homology, here used synonymously with identity, of 70, 75, 80, 85, 90, 92, 94, 96, 98, 99% or more percent, with respect to the corresponding original wildtype nucleic acid or amino acid sequence, wherein preferably the amino acids other than the amino acids forming the catalytically active centre or essential for the structure or folding are substituted or deleted or the latter are merely conservatively substituted, for example a glutamate in place of an aspartate or a leucine in place of a valine.

The prior art describes algorithms which may be used to calculate the degree of homology of two sequences, e.g. Arthur Lesk (2008), Introduction to Bioinformatics, 3^{rd} edition. In a further more preferred embodiment of the present invention, the variant of an amino acid or nucleic acid sequence, preferably in addition to the aforementioned sequence homology, has essentially the same enzymatic activity of the wildtype molecule and of the original molecule. For example, a variant of an enzymatically active protease polypeptide has the same, or essentially the same, proteolytic activity as the polypeptide enzyme, i.e. the capability to catalyse the hydrolysis of a peptide bond. In a particular embodiment, the term “essentially the same enzymatic activity” means an activity, with respect to the substrates of the wildtype polypeptide, which clearly lies above the background activity and/or differs from the K_{M} and/or k_{cat} values by less than 3, preferably 2, more preferably one, order of magnitude, which the wildtype polypeptide exhibits with respect to the same substrates. In a further preferred embodiment the term “variant” of a nucleic acid or amino acid sequence includes at least one active part/or fragment of the nucleic acid or amino acid sequence. In a further preferred embodiment, the term “active part”, as used here, means an amino acid sequence or a nucleic acid sequence which has less than the full length of the amino acid sequence and/or codes for less than the full length of the amino acid sequence, wherein the amino acid sequence or the coded amino acid sequence with a shorter length than the wildtype amino acid sequence essentially has the same enzymatic activity as the wildtype polypeptide or a variant thereof, for example, alcohol dehydrogenase, monooxygenase or transaminase. In a particular embodiment, the term “variant” of a nucleic acid comprises a nucleic acid whose complementary strand, preferably under stringent conditions, binds to the wildtype nucleic acid. The stringency of the hybridisation reaction is readily determinable by those skilled in the art and depends in general on the length of the probe, the washing temperatures and the salt concentration. Generally, longer probes require higher temperatures for the hybridisation, whereas shorter probes work at lower temperatures. Whether hybridisation takes place depends in general on the capability of the denatured DNA to anneal to complementary strands which are present in its environment and below the melting temperature. The stringency of hybridisation reactions and the corresponding conditions are described in more detail in Ausubel et al. 1995. In a preferred embodiment, the term “variant” of a nucleic acid, as used here, comprises any nucleic acid sequence which codes for the same amino acid sequence as the original nucleic acid or a variant of this amino acid sequence in terms of the degeneracy of the genetic code.

After the provision of isobutyric acid, this is, according to the invention, brought into contact with an enzyme or enzyme system, which has the capability to convert it to isobutyryl-CoA. It is possible to bring isobutyric acid into contact with the combination of isobutyrate kinase and phosphortransisobutyrylase. The term “isobutyrate kinase”, as used here, is understood to mean an enzyme which has the capability to phosphorylate isobutyric acid with hydrolysis of ATP. In a particularly preferred embodiment, the term “phosphotransisobutyrylase”, as used here, is understood to mean an enzyme that catalyses the conversion of the phosphorylated butyric acid to isobutyryl-CoA using coenzyme A. Those skilled in the art can find suitable enzymes in the prior art, for example: NP_{—}348286.1, YP_{—}001311072.1, YP_{—}001311673.1, YP_{—}003845108.1, CCC57671.1, ZP_{—}02993103.1, YP_{—}001255907.1, YP_{—}001788766.1, YP_{—}001783065.1, ZP_{—}02613551.1, ZP_{—}05129586.1, NP_{—}783068.1, ZP_{—}02616584.1, YP_{—}001392779.1, ZP_{—}02642313.1, YP_{—}697036.1, NP_{—}563263.1, YP_{—}699607.1, ZP_{—}05129585.1, ZP_{—}05394270.1, ZP_{—}04821992.1, YP_{—}001086582.1, YP_{—}001884532.1, YP_{—}001919732.1, YP_{—}001513941.1, YP_{—}001322041.1, NP_{—}349675.1, YP_{—}001307350.1, ZP_{—}02074622.1, AAA75487.1, ZP_{—}05979314.1, ZP_{—}02950703.1, YP_{—}003935519.1, YP_{—}001126403.1, ZP_{—}03147541.1, BAD11094.1, ZP_{—}08532470.1, CBK83142.1, ZP_{—}02027123.1, ZP_{—}02206646.1, ZP_{—}03226899.1, ZP_{—}05791023.1, YP_{—}002950277.1, YP_{—}003825217.1, ZP_{—}08609344.1, YP_{—}004819378.1, YP_{—}001376086.1, ZP_{—}06425397.1, ZP_{—}04152833.1, ZP_{—}03292064.1, ZP_{—}07525976.1, YP_{—}003477715.1, YP_{—}001664465.1, ZP_{—}04218902.1, YP_{—}003677577.1, ZP_{—}02211576.1, ZP_{—}04187800.1, ZP_{—}04302360.1, ZP_{—}04098303.1, ZP_{—}00394509.1, YP_{—}085496.1, NP_{—}846616.1, ZP_{—}04313571.1, YP_{—}896508.1, NP_{—}980529.1, ZP_{—}04170515.1, YP_{—}001646798.1, ZP_{—}00240356.1, ZP_{—}04269443.1, ZP_{—}04291055.1, ZP_{—}04176201.1, ZP_{—}01860365.1, ZP_{—}03237173.1, YP_{—}001471381.1, ZP_{—}08211979.1, ZP_{—}04208847.1, YP_{—}004820818.1, ZP_{—}04103879.1, YP_{—}003666354.1, NP_{—}833876.1, ZP_{—}04285820.1, ZP_{—}04086212.1, ZP_{—}04147476.1, NP_{—}623752.1, ZP_{—}03231885.1, ZP_{—}04066826.1, ZP_{—}08211238.1, ZP_{—}07709086.1, YP_{—}002315321.1, YP_{—}002447739.1, ZP_{—}00741048.1, ZP_{—}04073820.1, YP_{—}003988617.1, YP_{—}002368967.1, YP_{—}148233.1, YP_{—}003251443.1, AEN87678.1, YP_{—}003564885.1, YP_{—}004660977.1, YP_{—}004587379.1, NP_{—}349675.1, AAA75487.1, YP_{—}001788766.1, YP_{—}001255907.1, ZP_{—}02993103.1, YP_{—}001783065.1, ZP_{—}02613551.1, ZP_{—}02616584.1, YP_{—}001392779.1, ZP_{—}05394270.1, NP_{—}783068.1, CCC57671.1, YP_{—}697036.1, NP_{—}563263.1, YP_{—}699607.1, ZP_{—}02642313.1, YP_{—}001919732.1, ZP_{—}07525976.1, YP_{—}001884532.1, ZP_{—}04821992.1, ZP_{—}03292064.1, ZP_{—}05129586.1, YP_{—}001307350.1, ZP_{—}02950703.1, ZP_{—}05129585.1, YP_{—}001086582.1, YP_{—}001311072.1, YP_{—}001513941.1, YP_{—}003845108.1, YP_{—}001322041.1, ZP_{—}06425397.1, YP_{—}001311673.1, YP_{—}003935519.1, NP_{—}348286.1, ZP_{—}02027123.1, ZP_{—}08532470.1, YP_{—}001664465.1, YP_{—}002950277.1, YP_{—}004820818.1, ZP_{—}07709086.1, ZP_{—}08211979.1, ZP_{—}02211576.1, ZP_{—}05979314.1, ZP_{—}02074622.1, YP_{—}003677577.1, CBK83142.1, YP_{—}003477715.1, ZP_{—}02206646.1, BAD11094.1, ZP_{—}01860365.1, ZP_{—}03226899.1, NP_{—}623752.1, YP_{—}003988617.1, ZP_{—}08005605.1, YP_{—}003599607.1, YP_{—}003831638.1, YP_{—}003564885.1, YP_{—}004587379.1, AEN87678.1, YP_{—}001376086.1, YP_{—}004819378.1, ZP_{—}04152833.1, YP_{—}001126403.1, ZP_{—}03147541.1, ZP_{—}04218902.1, ZP_{—}08609344.1, ZP_{—}00240356.1, YP_{—}002368967.1, ZP_{—}04147476.1, ZP_{—}04176201.1, ZP_{—}04269443.1, YP_{—}003666354.1, ZP_{—}04073820.1, ZP_{—}04103879.1, NP_{—}833876.1, ZP_{—}04285820.1, ZP_{—}04187800.1, ZP_{—}04313571.1, ZP_{—}03231885.1, ZP_{—}03237173.1, ZP_{—}04086212.1, ZP_{—}04302360.1, ZP_{—}08094177.1, NP_{—}980529.1, ZP_{—}04098303.1, YP_{—}085496.1, ZP_{—}04170515.1, YP_{—}002447739.1, BAD11089.1, ZP_{—}00394509.1, YP_{—}001646798.1, YP_{—}002315321.1, NP_{—}846616.1, ZP_{—}04066826.1, YP_{—}896508.1, YP_{—}003866727.1, ZP_{—}00741048.1, YP_{—}001487375.1, ZP_{—}06875892.1, ZP_{—}04291055.1

Everything in this application relating to the sequence data from data bank codes from the prior art comes from the NCBI (National Center for Biotechnology Information, access date: 19 Oct. 2011) data bank, specifically using the release available online on 19 Oct. 2011.

Alternatively, isobutyryl-CoA may be obtained from isobutyric acid using isobutyryl-coenzyme A synthetase or ligase. In a preferred embodiment, the term “isobutyryl-coenzyme A ligase”, as used here, is understood to mean an enzyme that catalyses the conversion of isobutyric acid to isobutyryl-CoA using coenzyme A and nucleoside triphosphate. In a particularly preferred embodiment, the term “isobutyryl-coenzyme A synthetase”, as used here, is understood to mean an isobutyryl-coenzyme A ligase, wherein the NTP hydrolysed in the course of the reaction is ATP. Those skilled in the art can find suitable enzymes in the prior art, for example: NP_{—}579516.1, NP_{—}125992.1, YP_{—}004423263.1, YP_{—}004070968.1, YP_{—}182878.1, YP_{—}002306709.1, YP_{—}002959654.1, YP_{—}004762301.1, YP_{—}002581616.1, YP_{—}002994502.1, YP_{—}004623157.1, NP_{—}143577.1, YP_{—}002307387.1, NP_{—}579566.1, YP_{—}004623106.1, YP_{—}004763660.1, YP_{—}002583149.1, YP_{—}002959108.1, YP_{—}002993622.1, YP_{—}001736558.1, YP_{—}003649375.1, YP_{—}004423314.1, ZP_{—}04874839.1, YP_{—}183356.1, ZP_{—}04874991.1, YP_{—}001041242.1, YP_{—}003860266.1, NP_{—}126044.1, NP_{—}143628.1, YP_{—}003669211.1, YP_{—}002428622.1, YP_{—}001013511.1, YP_{—}004175933.1, YP_{—}004174284.1, YP_{—}001012369.1, YP_{—}004781520.1, YP_{—}004071372.1, NP_{—}769799.1, YP_{—}429257.1, CAJ70793.1, ZP_{—}08257475.1, YP_{—}930006.1, N1³_{—}618478.1, ZP_{—}08667208.1, YP_{—}001239140.1, YP_{—}004438172.1, YP_{—}003400029.1, YP_{—}001206872.1, NP_{—}070039.1, YP_{—}003727548.1, YP_{—}004625200.1, ZP_{—}08422040.1, YP_{—}460839.1, ZP_{—}08631067.1, ZP_{—}02883796.1, YP_{—}002953381.1, ZP_{—}08110577.1, YP_{—}003542795.1, ZP_{—}08905433.1, ZP_{—}06908198.1, YP_{—}001581540.1, NP_{—}632517.1, YP_{—}004519194.1, YP_{—}004515899.1, YP_{—}004120624.1, ZP_{—}07293826.1, CAJ73927.1, ZP_{—}07331643.1, YP_{—}001278815.1, ZP_{—}08840735.1, YP_{—}002289530.1, YP_{—}001637486.1, YP_{—}004342727.1, ZP_{—}07026137.1, ZP_{—}08112481.1, YP_{—}003487360.1, YP_{—}001540910.1, ZP_{—}07946223.1, YP_{—}685524.1, YP_{—}004812635.1, ZP_{—}08677213.1, ZP_{—}08803651.1, ADI05837.1, YP_{—}874976.1, YP_{—}002465105.1, YP_{—}003355503.1, ZP_{—}07026765.1, YP_{—}001430231.1, YP_{—}004893707.1, YP_{—}003766745.1, YP_{—}004627663.1, YP_{—}003649567.1, ZP_{—}07307686.1, YP_{—}001546514.1, YP_{—}686303.1, YP_{—}002992636.1, YP_{—}004516207.1, YP_{—}001154008.1, YP_{—}004338243.1, YP_{—}003357973.1

Lastly, isobutyryl-CoA may be prepared from isobutyric acid by means of isobutyrate-coenzyme A transferase. In a preferred embodiment, the term “isobutyrate-coenzyme A transferase”, as used here, is an enzyme that catalyses the formation of isobutyryl-CoA from isobutyric acid by transferring coenzyme A from an acyl-CoA functioning as donor. Those skilled in the art can find suitable enzymes in the prior art, for example: NP_{—}149326.1, AAB53234.1, YP_{—}001310904.1, AAD54947.1, AAP42564.1, CAQ57984.1, YP_{—}001886322.1, NP_{—}622378.1, ZP_{—}08693244.1, ZP_{—}07926619.1, ZP_{—}08555875.1, ZP_{—}04390377.1, ZP_{—}01867058.1, ZP_{—}04573915.1, ZP_{—}07913714.1, ZP_{—}07923474.1, ZP_{—}08691337.1, ZP_{—}08600063.1, ZP_{—}02692961.1, ZP_{—}08582386.1, ZP_{—}00144733.1, ZP_{—}05815087.1, ZP_{—}06524353.1, ZP_{—}07952599.1, YP_{—}004254308.1, YP_{—}003039857.1, YP_{—}003828410.1, ZP_{—}06175535.1, NP_{—}602657.1, ZP_{—}06748826.1, ZP_{—}06749807.1, ZP_{—}04970682.1, ADO77683.1, AAO18070.1, ZP_{—}05887867.1, AEJ99145.1, EGB63075.1, NP_{—}931005.1, YP_{—}003968227.1, ZP_{—}08327315.1, ZP_{—}06025832.1, YP_{—}003260518.1, YP_{—}003016746.1, YP_{—}001452140.1, EGW68380.1, ZP_{—}02424926.1, ZP_{—}03828051.1, EGB72667.1, EFW53769.1, ZP_{—}03001766.1, ZP_{—}05402063.1, YP_{—}049390.1, ZP_{—}08690265.1, YP_{—}003936148.1, YP_{—}002382110.1, NP_{—}416725.1, YP_{—}003941041.1, YP_{—}001744415.1, YP_{—}003296165.1, ZP_{—}07151665.1, NP_{—}754650.1, YP_{—}002413271.1, EGC06740.1, YP_{—}001459023.1, ZP_{—}05272741.1, ZP_{—}04005084.1, YP_{—}541501.1, YP_{—}002335226.1, YP_{—}001089188.1, ZP_{—}04054428.1, YP_{—}001784143.1, YP_{—}001306376.1, ZP_{—}07820010.1, ZP_{—}03065638.1, YP_{—}004441693.1, NP_{—}905281.1, YP_{—}001321984.1, ZP_{—}06636026.1, YP_{—}003296060.1, ZP_{—}07903700.1, YP_{—}004509865.1, ZP_{—}08626421.1, YP_{—}002933615.1, YP_{—}002771575.1, ZP_{—}06715088.1, ZP_{—}01548307.1, ZP_{—}06982396.1, ZP_{—}08643079.1, YP_{—}002497558.1, YP_{—}001513889.1, NP_{—}438933.1, YP_{—}248482.1, YP_{—}531878.1, YP_{—}002746872.1, YP_{—}002744076.1, ZP_{—}01792337.1, YP_{—}002940319.1, YP_{—}001471175.1, ZP_{—}01220381.1, YP_{—}002123783.1, NP_{—}149327.1, CAQ57985.1, YP_{—}001886321.1, AAD54948.1, YP_{—}001310905.1, AAP42565.1, ZP_{—}05092257.1, NP_{—}622379.1, ZP_{—}08555876.1, ZP_{—}02692960.1, YP_{—}002335225.1, YP_{—}001306375.1, ZP_{—}08693245.1, YP_{—}001513888.1, YP_{—}001321983.1, ZP_{—}07926620.1, ZP_{—}02424916.1, YP_{—}001409735.1, ZP_{—}06983458.1, YP_{—}003828409.1, YP_{—}004707898.1, YP_{—}003936149.1, ZP_{—}07577382.1, YP_{—}001089189.1, YP_{—}001471174.1, ZP_{—}05272742.1, YP_{—}002940318.1, ZP_{—}05402064.1, YP_{—}004254317.1, YP_{—}003968226.1, CBK81879.1, ZP_{—}07819217.1, YP_{—}001740168.1, YP_{—}049389.1, ZP_{—}08709179.1, YP_{—}001918401.1, YP_{—}003016745.1, NP_{—}905290.1, YP_{—}004509856.1, ZP_{—}03828050.1, YP_{—}426559.1, YP_{—}001929287.1, YP_{—}001568118.1, ZP_{—}04390271.1, ZP_{—}07913715.1, ZP_{—}07820042.1, YP_{—}003260519.1, ZP_{—}08691338.1, ZP_{—}01548308.1, ZP_{—}08327314.1, ZP_{—}04970681.1, ZP_{—}04054413.1, ZP_{—}08690266.1, ZP_{—}05887868.1, ZP_{—}06748825.1, YP_{—}001918068.1, ZP_{—}08731713.1, NP_{—}602656.1, ZP_{—}06524354.1, ZP_{—}01867057.1, ZP_{—}04573916.1, ZP_{—}05815086.1, ZP_{—}06025831.1, ZP_{—}00144734.1, ZP_{—}08600062.1, YP_{—}001784144.1, EGQ80092.1, ZP_{—}06175534.1, ADO77682.1, ZP_{—}01220382.1, YP_{—}597732.1, YP_{—}003945474.1, YP_{—}004219013.1, YP_{—}002562694.1, YP_{—}001788168.1, YP_{—}002285170.1, NP_{—}268527.1, YP_{—}001392125.1, CBZ04738.1, ZP_{—}06921170.1, ZP_{—}07461446.1, YP_{—}531879.1, YP_{—}002805353.1, YP_{—}003452384.1, YP_{—}001255346.1, YP_{—}001385091.1, YP_{—}878775.1, EGC06741.1, NP_{—}663914.1, YP_{—}059486.1, YP_{—}002382109.1, YP_{—}001884900.1, ZP_{—}07307813.1, ZP_{—}02621896.1, ZP_{—}07903699.1, ZP_{—}08729700.1, YP_{—}001309762.1, YP_{—}001768507.1, YP_{—}602949.1, EGL48602.1, XP_{—}501388.1, XP_{—}002841479.1, XP_{—}002481641.1, XP_{—}002147496.1, XP_{—}001879757.1, XP_{—}661332.1, XP_{—}003295652.1, XP_{—}001936644.1, AAK40365.1, XP_{—}001802255.1, XP_{—}002383405.1, XP_{—}001390976.1, XP_{—}001212457.1, EGP83670.1, XP_{—}001227675.1, XP_{—}003176313.1, XP_{—}001268294.1, XP_{—}751117.1, EGF82407.1, XP_{—}001258382.1, EFW13413.1, XP_{—}003065510.1, EGD93452.1, EEH22314.1, XP_{—}001247983.1, EGE09225.1, XP_{—}003022194.1, CBX98353.1, XP_{—}003014185.1, XP_{—}002564448.1, XP_{—}003239146.1, EGX51025.1, EGO01287.1, XP_{—}002792936.1, EFQ30503.1, XP_{—}001838038.1, XP_{—}002847737.1, EGO55093.1, XP_{—}957979.1, XP_{—}001547410.1, EGR48848.1, XP_{—}003038014.1, EER44122.1, EFY99528.1, EGC42434.1, EEH09119.1, EFY92418.1, EGS22244.1, XP_{—}002629107.1, XP_{—}002548743.1, XP_{—}001904655.1, EFX03923.1, XP_{—}716276.1, XP_{—}002422356.1, XP_{—}388121.1, XP_{—}001586993.1, EGX96527.1, XP_{—}368380.1, NP_{—}595848.1, XP_{—}003345762.1, XP_{—}759662.1, XP_{—}003001963.1, EGG06029.1, XP_{—}002175297.1, XP_{—}001386078.2, XP_{—}003040746.1, XP_{—}459426.2, EDK36000.2, CBQ73919.1, EGU85384.1, XP_{—}001525161.1, XP_{—}001731221.1, XP_{—}002490853.1, XP_{—}001486721.1

The next step in the biotechnological synthesis of 3-hydroxyisobutyric acid according to the invention comprises bringing the product from step b), the isobutyryl-CoA, into contact with an isobutyryl-coenzyme A dehydrogenase. In a preferred embodiment, the term “isobutyryl-coenzyme A dehydrogenase”, as used here, is understood to mean an enzyme that catalyses the oxidation of isobutyryl-coenzyme A to methacrylyl-coenzyme A with release of reduction equivalents. Suitable examples include the polypeptides known from the prior art with the databank codes XP_{—}501919.2, EDP50227.1, XP_{—}001267173.1, XP_{—}751977.1, EFW17827.1, XP_{—}001241675.1, XP_{—}003070631.1, XP_{—}002376988.1, EGS22147.1, XP_{—}001271742.1, XP_{—}002794645.1, XP_{—}001214528.1, XP_{—}959931.1, EGO55678.1, EEH46977.1, XP_{—}002543210.1, XP_{—}001401697.1, EEH07104.1, BAE59223.1, EGU83504.1, XP_{—}002627767.1, XP_{—}002483661.1, EFX03379.1, XP_{—}003048205.1, EGX54111.1, XP_{—}002150528.1, EFQ35634.1, XP_{—}001548029.1, XP_{—}659303.1, XP_{—}002841387.1, XP_{—}001791413.1, XP_{—}001904778.1, XP_{—}390966.1, XP_{—}003015640.1, EGD94205.1, XP_{—}003233609.1, XP_{—}003018507.1, XP_{—}002561081.1, XP_{—}360875.1, EFY92504.1, XP_{—}003344493.1, CBX95087.1, EGC41158.1, EGX92432.1, XP_{—}003169516.1, EFZ00003.1, EGR49157.1, EGP87134.1, XP_{—}002849756.1, XP_{—}003302688.1, XP_{—}001937377.1, EEH18077.1, XP_{—}001227538.1, XP_{—}001586947.1, AAK63186.1, XP_{—}001538624.1, AAQ04622.1, EGF84480.1, EER45206.1, XP_{—}502873.1, EGS21840.1, XP_{—}752854.1, XP_{—}003170973.1, CBF88712.1, XP_{—}001264273.1, XP_{—}003232243.1, EGD97329.1, XP_{—}002479178.1, XP_{—}658428.1, XP_{—}002844533.1, EGE05996.1, XP_{—}001223344.1, EGX95352.1, XP_{—}001389698.2, CAK37343.1, EGR51332.1, XP_{—}003068884.1, XP_{—}002146883.1, XP_{—}001243830.1, CAP65331.1, XP_{—}001268770.1, XP_{—}001818195.1, EFQ34732.1, XP_{—}001210950.1, XP_{—}962250.1, EEHSO726.1, XP_{—}001804465.1, XP_{—}003023750.1, EGO58886.1, CBX97678.1, XP_{—}002561648.1, EEH15844.1, XP_{—}389837.1, XP_{—}363106.2, EFY97392.1, EFY90016.1, EGU81915.1, XP_{—}002621381.1, EEQ89840.1, EGX53945.1, XP_{—}003295397.1, XP_{—}003011911.1, EER38173.1, XP_{—}002792725.1, XP_{—}003045885.1, XP_{—}001935173.1, EGP88451.1, XP_{—}001584930.1, XP_{—}001836878.1, EGG04035.1, XP_{—}003005525.1, XP_{—}001553916.1, XP_{—}762332.1, CBQ71452.1, EFX05387.1, XP_{—}568632.1, XP_{—}001884381.1, EGO01481.1, XP_{—}003332014.1, XP_{—}003197572.1, XP_{—}001731213.1, XP_{—}003026264.1, EGF80206.1, XP_{—}003346492.1, XP_{—}003324899.1, EEH09831.1, XP_{—}002582344.1, EGT41105.1, XP_{—}002640134.1, CBJ32167.1, XP_{—}002607968.1, EFW43327.1, XP_{—}003099748.1, CAF95757.1, NP_{—}491859.1, XP_{—}002471089.1, YP_{—}001611803.1, XP_{—}002904727.1, EGG17601.1, EFX71478.1, CBN81547.1, XP_{—}002640162.1, ADY46184.1, XP_{—}003099703.1, EFA76871.1, AAH82665.1, 2JIF_A, XP_{—}001658431.1, CAD38535.2, AAH13756.1, NP_{—}001124722.1, NP_{—}001600.1, XP_{—}003255101.1, XP_{—}003283361.1, CAJ81939.1, XP_{—}002640145.1, NP_{—}491871.1, XP_{—}001104844.1, AAH54428.1

The next step of the method according to the invention comprises the hydration of methacrylyl-coenzyme A to form 3-hydroxyisobutyryl-coenzyme A by means of a methacrylyl-coenzyme A hydratase. In a preferred embodiment, the term “methacrylyl-coenzyme A hydratase”, as used here, is understood to mean an enzyme that catalyses the addition of a water molecule onto methacrylyl-coenzyme A to form 3-hydroxyisobutyric acid. Examples include the polypeptides known from the prior art with the databank codes XP_{—}502475.1, XP_{—}003067220.1, XP_{—}001239658.1, XP_{—}002567879.1, XP_{—}002145078.1, XP_{—}001259415.1, CAK97202.1, XP_{—}001211164.1, XP_{—}001401252.2, XP_{—}753374.1, XP_{—}664448.1, CBF71576.1, XP_{—}001274572.1, XP_{—}002340305.1, XP_{—}002845201.1, XP_{—}001824127.1, XP_{—}002795848.1, XP_{—}003304302.1, EGC44435.1, EER38804.1, EEH09966.1, XP_{—}001936195.1, EEHSO797.1, EEH15784.1, EGO61149.1, XP_{—}003170409.1, XP_{—}961123.1, EGS18121.1, XP_{—}001554659.1, XP_{—}003230996.1, EGE07573.1, XP_{—}002621253.1, EGE81015.1, EEQ89969.1, XP_{—}361538.2, XP_{—}001224889.1, XP_{—}002381216.1, CBX96718.1, EFY95366.1, XP_{—}001595164.1, XP_{—}001911969.1, EGX51365.1, EFY87978.1, EFQ29854.1, EGU81865.1, XP_{—}003050469.1, XP_{—}003352189.1, XP_{—}002839588.1, XP_{—}003002061.1, EGD96635.1, XP_{—}003022976.1, XP_{—}003010670.1, EFX03685.1, EGR47862.1, XP_{—}002584325.1, XP_{—}387195.1, EGX94938.1, XP_{—}002616791.1, XP_{—}462069.2, XP_{—}001800531.1

Finally, the hydrolysis of the product from step d), the 3-hydroxyisobutyryl-CoA, is required to release the desired product, 3-hydroxyisobutyric acid. For this purpose, the possibility exists initially to subject the product to extreme pH conditions by addition of acid or base, which promote the hydrolysis without the action of a further enzyme. In a particularly preferred embodiment, the hydrolysis takes place however by bringing the product from step c) into contact with a 3-hydroxyisobutyryl-coenzyme A hydrolase. In a preferred embodiment, the term “3-hydroxyisobutyryl-coenzyme A hydrolase”, as used here, is understood to mean an enzyme which hydrolyses 3-hydroxyisobutyryl-coenzyme A to 3-hydroxyisobutyric acid and coenzyme A. The following proteins may be cited here as examples: XP_{—}504911.1, XP_{—}003066853.1, XP_{—}001246264.1, XP_{—}385460.1, EFY92465.1, XP_{—}363761.1, XP_{—}003346508.1, EFY97405.1, EEQ90955.1, XP_{—}002623105.1, XP_{—}001223375.1, XP_{—}002794385.1, XP_{—}003046454.1, EGO58901.1, EEH47368.1, EGS21819.1, EEH18429.1, AAK07843.1, XP_{—}002540650.1, XP_{—}002484211.1, XP_{—}002150057.1, EFQ36202.1, EDP49675.1, XP_{—}750988.1, XP_{—}003235672.1, EGR51338.1, XP_{—}003013762.1, XP_{—}003019423.1, EGE05569.1, XP_{—}003169276.1, EGD98503.1, EEH06901.1, XP_{—}001544047.1, EGC47721.1, XP_{—}001821058.1, EGE82172.1, XP_{—}002842837.1, XP_{—}002835633.1, EGX53716.1, EFX05960.1, XP_{—}001590627.1, XP_{—}001552254.1, XP_{—}962266.2, EGX95378.1, XP_{—}001392906.1, XP_{—}003302932.1, CBF89164.1, EGU77782.1, EGP84417.1, XP_{—}001258223.1, XP_{—}001268201.1, XP_{—}002569077.1, XP_{—}001214240.1, EER38573.1, ADD19825.1, XP_{—}001939164.1, XP_{—}658197.1, XP_{—}001248657.1, XP_{—}315590.3, XP_{—}003047713.1, EFR21351.1, EGP83804.1, EFX82586.1, EFN83706.1, XP_{—}001799030.1, XP_{—}002423077.1, XP_{—}002073789.1, XP_{—}001987737.1, XP_{—}002050275.1, XP_{—}002091863.1, XP_{—}002005175.1, XP_{—}001974736.1, EGF81169.1, EFW47689.1, XP_{—}002149358.1, XP_{—}002149354.1, CBY01415.1, NP_{—}611373.1, CAP65353.1, XP_{—}002569208.1, AAL39202.2, XP_{—}002543763.1, XP_{—}001664110.1, XP_{—}002034506.1, XP_{—}002565243.1, XP_{—}003049329.1, XP_{—}001360407.1, XP_{—}001865614.1

Biologically active enzymes are used, according to the invention, in steps b) to d). In this connection, as long as at least one of the enzymes is used in the form of a cell which, compared to its wildtype, has a reduced activity of a 3-hydroxyisobutyric acid dehydrogenase or a variant thereof, as in the case of all enzymatically active polypeptides used according to the invention, these may be cells comprising enzymatically active polypeptides or their lysates or preparations of the polypeptides at all purification levels, from the intact cell or its crude lysate up to the pure polypeptide, which have the respective biologically active enzyme in endogenous or recombinant form, preferably overexpressed. In this field, numerous methods are known to those skilled in the art with which enzymatically active polypeptides can be overexpressed in suitable cells and purified or isolated. All expression systems available to those skilled in the art can thus be used for the expression of the polypeptides, for example, vectors of the pET type or pGEX type. Chromatographic methods are suitable for purification, for example affinity chromatography purification of a recombinant protein provided with a for example tag by using an immobilised ligand, for example, a nickel ion in the case of a histidine tag, of immobilised glutathione in the case of a glutathione S-transferase fusioned onto the target protein or of immobilised maltose in the case of a tag containing maltose-binding protein. Those skilled in the art are also aware how they can work out suitable reaction conditions in the context of their routine experimentation, under which the enzyme of interest shows activity, preferably optimal activity. These conditions include, for example, the selection of suitable buffers, the evaluation and adjustment of the optimal pH, a specific salt concentration and specific minimum protein concentration; see, for example, Cornish-Bowden, 1995.

If purified enzymes are used for the method according to the invention and not intact living cells, then the former may be used either in soluble form or immobilised. Suitable methods are known to those skilled in the art by which polypeptides may be covalently or non-covalently immobilised on organic or inorganic solid phases, for example by sulphydryl-coupling chemistry (e.g. kits from the Pierce company).

Since the method according to the invention however makes use of a plurality of enzymes with different cofactors, which possibly are added in stoichiometric amounts in the case of purified polypeptides, the enzymes required for the method are provided, in a particularly preferred embodiment, in the form of a single whole-cell catalyst, i.e. in the form of a viable, metabolically active cell. The enzymes may be presented on the surface of the whole-cell catalyst, as described in the prior art, for example in DE 60216245. However, it is very particularly preferred that the enzymes dependent on regenerating cofactors, more preferably all enzymes, are localised in such a way that their active centres remain in contact with the interior of the cell, so that the required cofactors and cosubstrates are acquired from the cell metabolism and further resupplied.

The preparation of mutants of a cell which has a specific enzymatic activity, with the aim of reducing this enzymatic activity for the mutants to be obtained compared to the wildtype of the cell, is feasible to those skilled in the art using standard methods in the field of molecular biology, genetics and microbiology (Sambrook et al., 1989). For example, a random mutagenesis by treatment of wildtype cells with radioactive radiation followed by a step for selecting suitable mutants by determining the enzymatic activity of isolated colonies using suitable assays is possible, as described for numerous enzymes in the prior art (Cornish-Bowden, 1995). Further methods comprising the insertion of a deactivating point mutations, for example into the promoter or into the active centre of the enzymatically active polypeptide, is a method also established for decades (Fersht and Winter, 2008).

In a preferred embodiment, the cell used is a prokaryotic, preferably a bacterial, cell. In a further preferred embodiment, it is a mammalian cell. In a further preferred embodiment, it is a lower eukaryotic cell, preferably a yeast cell. Examples of prokaryotic cells include Escherichia, particularly Escherichia coli, and strains of the genus Pseudomonas and Corynebacterium. Examples of lower eukaryotic cells include the genera Saccharomyces, Candida, Pichia, Yarrowia, Schizosaccharomyces, particularly the strains Candida tropicalis, Schizosaccharomyces pombe, Pichia pastoris, Yarrowia lipolytica and Saccharomyces cerivisiae. In the most preferred embodiment, it is Yarrowia lipolytica.

An aspect essential to the teaching according to the invention consists in the fact that a cell is used whose 3-hydroxyisobutyric acid dehydrogenase activity, or the activity of a variant thereof, is reduced. In a preferred embodiment, the term “3-hydroxyisobutyric acid dehydrogenase activity”, as used here, is understood to mean the activity of an enzyme that oxidises 3-hydroxyisobutyric acid to the aldehyde.

In a preferred embodiment, the 3-hydroxyisobutyric acid dehydrogenase is an enzyme from the group which comprises the polypeptides known from the prior art XP_{—}504911.1, XP_{—}003066853.1, XP_{—}001246264.1, XP_{—}385460.1, EFY92465.1, XP_{—}363761.1, XP_{—}003346508.1, EFY97405.1, EEQ90955.1, XP_{—}002623105.1, XP_{—}001223375.1, XP_{—}002794385.1, XP_{—}003046454.1, EGO58901.1, EEH47368.1, EGS21819.1, EEH18429.1, AAK07843.1, XP_{—}002540650.1, XP_{—}002484211.1, XP_{—}002150057.1, EFQ36202.1, EDP49675.1, XP_{—}750988.1, XP_{—}003235672.1, EGR51338.1, XP_{—}003013762.1, XP_{—}003019423.1, EGE05569.1, XP_{—}003169276.1, EGD98503.1, EEH06901.1, XP_{—}001544047.1, EGC47721.1, XP_{—}001821058.1, EGE82172.1, XP_{—}002842837.1, XP_{—}002835633.1, EFX05960.1, XP_{—}001590627.1, XP_{—}001552254.1, XP_{—}962266.2, XP_{—}001392906.1, XP_{—}003302932.1, CBF89164.1, EGP84417.1, XP_{—}001258223.1, XP_{—}001268201.1, XP_{—}002569077.1, XP_{—}001214240.1, EER38573.1, XP_{—}001939164.1, XP_{—}658197.1, XP_{—}001248657.1, XP_{—}003047713.1, EGP83804.1, XP_{—}001799030.1, EGF81169.1, XP_{—}002149358.1, XP_{—}002149354.1, CBY01415.1, CAP65353.1, XP_{—}002569208.1, XP_{—}002543763.1, XP_{—}002565243.1, XP_{—}003049329.1, XP_{—}001933034.1, XP_{—}002551090.1, XP_{—}001209304.1, XP_{—}001796105.1, XP_{—}003305815.1, CBY01417.1, XP_{—}002144318.1, XP_{—}003035025.1, XP_{—}003047232.1, EDK39415.2, XP_{—}003070936.1, XP_{—}001878640.1, XP_{—}001833463.1, XP_{—}001484132.1, XP_{—}003051032.1, XP_{—}719244.1, XP_{—}001796108.1, XP_{—}456589.2, XP_{—}001384410.2, XP_{—}002421055.1, XP_{—}719127.1, XP_{—}001524095.1, XP_{—}003336106.1, EGN96107.1, XP_{—}003043164.1, XP_{—}001903154.1, XP_{—}758336.1, XP_{—}003051445.1, CBX93804.1, XP_{—}002614534.1, EFW22385.1, XP_{—}003047715.1, XP_{—}003009661.1, XP_{—}001903159.1, XP_{—}002144320.1, XP_{—}754672.2, EGG05569.1, more preferably is an enzyme from the group XP_{—}504911.1, XP_{—}003066853.1, XP_{—}001246264.1, XP_{—}385460.1, EFY92465.1, XP_{—}363761.1, XP_{—}003346508.1, EFY97405.1, EEQ90955.1, XP_{—}002623105.1, XP_{—}001223375.1, XP_{—}002794385.1, XP_{—}003046454.1, EGO58901.1, EEH47368.1, EGS21819.1, EEH18429.1, AAK07843.1, XP_{—}002540650.1, XP_{—}002484211.1, XP_{—}002150057.1, EFQ36202.1, EDP49675.1, XP_{—}750988.1, XP_{—}003235672.1, EGR51338.1, XP_{—}003013762.1, XP_{—}003019423.1, EGE05569.1, XP_{—}003169276.1, EGD98503.1, EEH06901.1, XP_{—}001544047.1, EGC47721.1, XP_{—}001821058.1, EGE82172.1, XP_{—}002842837.1, XP_{—}002835633.1, EFX05960.1, XP_{—}001590627.1, XP_{—}001552254.1, XP_{—}962266.2, XP_{—}001392906.1, XP_{—}003302932.1, CBF89164.1, EGP84417.1, XP_{—}001258223.1, XP_{—}001268201.1, XP_{—}002569077.1, XP_{—}001214240.1, EER38573.1, XP_{—}001939164.1, XP_{—}658197.1, XP_{—}001248657.1, XP_{—}003047713.1, XP_{—}002551090.1, EDK39415.2, XP_{—}001484132.1, XP_{—}719244.1, XP_{—}456589.2, XP_{—}001384410.2, XP_{—}002421055.1, XP_{—}719127.1, XP_{—}001524095.1, XP_{—}002614534.1, XP_{—}504911.1, particularly preferably is XP_{—}504911.1 XP_{—}002551090.1, EDK39415.2, XP_{—}001484132.1, XP_{—}719244.1, XP_{—}456589.2, XP_{—}001384410.2, XP_{—}002421055.1, XP_{—}719127.1, XP_{—}001524095.1, XP_{—}002614534.1, and most prefarably is XP_{—}504911.1.

In a preferred embodiment, the 3-hydroxyisobutyric acid dehydrogenase is a 3-hydroxyisobutyric acid dehydrogenase from the group which comprises the polypeptides known from the prior art BAC82381.1, NP_{—}746775.1, YP_{—}004703920.1, YP_{—}001670886.1, ADR61938.1, YP_{—}001269834.1, YP_{—}001747642.1, YP_{—}606441.1, BAJ07617.1, YP_{—}257885.1, EGH62730.1, ZP_{—}06461142.1, ZP_{—}05642104.1, YP_{—}004351842.1, EGH86869.1, YP_{—}002874705.1, EGH24275.1, ZP_{—}07777517.1, ZP_{—}07003462.1, ZP_{—}07264531.1, EGH72043.1, YP_{—}233798.1, EGH66664.1, EGH33025.1, EGH77479.1, EGH54475.1, EFW86611.1, EGH13085.1, ZP_{—}03399204.1, NP_{—}790630.1, YP_{—}346429.1, ZP_{—}04590292.1, EGH46911.1, YP_{—}004681354.1, YP_{—}840711.1, YP_{—}002007768.1, YP_{—}298456.1, ZP_{—}06495709.1, YP_{—}001777122.1, YP_{—}837675.1, YP_{—}624179.1, YP_{—}001117046.1, YP_{—}555519.1, YP_{—}004360017.1, ZP_{—}07673810.1, YP_{—}004714189.1, YP_{—}003607711.1, YP_{—}557465.1, YP_{—}001810630.1, YP_{—}003749513.1, ZP_{—}08140457.1, AEA83842.1, YP_{—}001861262.1, YP_{—}001892508.1, ZP_{—}03264187.1, ADY83615.1, YP_{—}001844789.1, YP_{—}002233766.1, ZP_{—}02893156.1, ZP_{—}06693498.1, YP_{—}001705868.1, YP_{—}001715485.1, YP_{—}775329.1, ADX90568.1, ZP_{—}06059137.1, ADX01770.1, ZP_{—}05825497.1, ZP_{—}06069662.1, YP_{—}372791.1, YP_{—}002254778.1, AEG71628.1, YP_{—}001584514.1, ZP_{—}02358230.1, ZP_{—}03569839.1, YP_{—}439994.1, ZP_{—}02365286.1, YP_{—}554218.1, YP_{—}004473140.1, YP_{—}001889364.1, ZP_{—}03585594.1, YP_{—}110641.1, YP_{—}003734018.1, ZP_{—}06839562.1, YP_{—}001583635.1, ZP_{—}00944342.1, ZP_{—}03573494.1, YP_{—}001115788.1, ZP_{—}03582471.1, YP_{—}776268.1, YP_{—}623275.1, ZP_{—}04522920.1, ZP_{—}04947734.1, ZP_{—}02910304.1, YP_{—}371475.1, YP_{—}001811564.1, YP_{—}002798276.1, YP_{—}003910785.1, YP_{—}004230293.1, ZP_{—}02881368.1, ZP_{—}03822695.1, ZP_{—}02891780.1, ZP_{—}06726280.1, CBJ40252.1, ZP_{—}02376988.1, ZP_{—}05359923.1, YP_{—}002908300.1, YP_{—}004348870.1, YP_{—}001172440.1, YP_{—}003747923.1, YP_{—}046276.1, NP_{—}522210.1, YP_{—}001156335.1, ZP_{—}03268101.1, ZP_{—}07236476.1, YP_{—}001861075.1, ZP_{—}05136769.1, YP_{—}293155.1, YP_{—}001083192.1, YP_{—}001970187.1, YP_{—}002026611.1, YP_{—}003776462.1, ZP_{—}02381313.1, ADP96674.1, EGF41785.1, ZP_{—}08410855.1, ZP_{—}05911432.1, AEL06416.1, ZP_{—}01989307.1, ZP_{—}06178628.1, NP_{—}636638.1, ZP_{—}01260019.1, ZP_{—}04929809.1, ZP_{—}08460617.1, YP_{—}004068587.1, YP_{—}002553266.1, ZP_{—}06877402.1, YP_{—}002439068.1, NP_{—}800628.1, ZP_{—}08100296.1, NP_{—}252259.1, YP_{—}001279202.1, ZP_{—}01367000.1, YP_{—}001346959.1, ZP_{—}01133887.1, EGM15746.1, YP_{—}339977.1, ZP_{—}04921053.1, ZP_{—}04764368.1, NP_{—}762450.2, YP_{—}986257.1, YP_{—}789596.1, YP_{—}004191179.1, YP_{—}004416219.1, NP_{—}937094.1, YP_{—}002794225.1, ZP_{—}02244049.1, ZP_{—}05943914.1, YP_{—}450771.1, YP_{—}200485.1, ZP_{—}08178115.1, NP_{—}717293.1, ZP_{—}06730192.1, ZP_{—}04958952.1, ZP_{—}03698449.1, ZP_{—}04922804.1, YP_{—}003288698.1, YP_{—}001141739.1, ZP_{—}08188278.1, YP_{—}786279.1, ZP_{—}05127790.1, YP_{—}363098.1, ZP_{—}01104144.1, ZP_{—}05886382.1, ZP_{—}05620602.1, ZP_{—}01614260.1, NP_{—}641651.1, ZP_{—}06705559.1, ZP_{—}01260775.1, ZP_{—}06182137.1, ZP_{—}05879122.1, YP_{—}001631119.1, ZP_{—}08552251.1, ZP_{—}08570660.1, ZP_{—}01738211.1, ZP_{—}01893199.1, ZP_{—}08520681.1, ZP_{—}08182597.1, YP_{—}264191.1, YP_{—}001414164.1, ZP_{—}08638362.1, YP_{—}422950.1, EGF41343.1, ZP_{—}00056040.1, ZP_{—}08700476.1, NP_{—}800135.1, ZP_{—}08272093.1, ZP_{—}08181910.1, ZP_{—}02194300.1, ZP_{—}03698989.1, YP_{—}004235082.1, preferably is an enzyme from the group comprising the enzymes BAC82381.1, NP_{—}746775.1, YP_{—}004703920.1, YP_{—}001670886.1, ADR61938.1, YP_{—}001269834.1, YP_{—}001747642.1, YP_{—}606441.1, BAJ07617.1, YP_{—}257885.1, EGH62730.1, ZP_{—}06461142.1, ZP_{—}05642104.1, YP_{—}004351842.1, EGH86869.1, YP_{—}002874705.1, EGH24275.1, ZP_{—}07777517.1, ZP_{—}07003462.1, ZP_{—}07264531.1, EGH72043.1, YP_{—}233798.1, EGH66664.1, EGH33025.1, EGH77479.1, EGH54475.1, EFW86611.1, EGH13085.1, ZP_{—}03399204.1, NP_{—}790630.1, YP_{—}346429.1, ZP_{—}04590292.1, EGH46911.1, ZP_{—}06495709.1, YP_{—}004714189.1, ZP_{—}08140457.1, AEA83842.1, ADY83615.1, YP_{—}001844789.1, ZP_{—}06693498.1, YP_{—}001705868.1, YP_{—}001715485.1, ADX90568.1, ZP_{—}06059137.1, ADX01770.1, ZP_{—}05825497.1, ZP_{—}06069662.1, YP_{—}004473140.1, YP_{—}003734018.1, YP_{—}002798276.1, ZP_{—}03822695.1, ZP_{—}06726280.1, ZP_{—}05359923.1, YP_{—}001172440.1, YP_{—}046276.1, ZP_{—}07236476.1, YP_{—}001083192.1, ZP_{—}04929809.1, ZP_{—}08460617.1, ZP_{—}06877402.1, YP_{—}002439068.1, NP_{—}252259.1, YP_{—}001279202.1, ZP_{—}01367000.1, YP_{—}001346959.1, EGM15746.1, YP_{—}789596.1, ZP_{—}05620602.1, YP_{—}264191.1, YP_{—}580776.1, YP_{—}792542.1, YP_{—}004379211.1, ZP_{—}06880384.1, YP_{—}001186868.1, ZP_{—}04932430.1, ZP_{—}01364121.1, YP_{—}002442182.1, NP_{—}249434.1, YP_{—}001350119.1, EGH98403.1, 3OBB_A, 3Q3C_A. In a preferred embodiment, the phrase “a cell which, compared to its wildtype, has a reduced activity of a 3-hydroxyisobutyric acid dehydrogenase or a variant thereof” means that it is a cell which is genetically modified compared to its wildtype in such a way that the activity of a 3-hydroxyisobutyric acid dehydrogenase, or a variant, for this cell compared to the activity of the identical 3-hydroyxisobutyric acid dehydrogenase or of the corresponding variant for the wildtype of the cell is reduced, preferably by at least 10, 20, 30, 50, 75, 90, 95 or 99%. In a particularly preferred embodiment, the activity of the enzyme for the genetically modified cell is no longer detectable. In a further particularly preferred embodiment, the genetic modification causing the reduction in activity refers exclusively to the activity of a specific 3-hydroxyisobutyric acid dehydrogenase of the cell, in contrast to the possibility that several non-specific enzymatic activities of the cell are reduced, for example through a defect in the folding mechanisms of the cell, which might cause numerous enzymatic activities of the cell to be defective. A comparison of the enzymatic activities of the genetically modified cell and its wildtype takes place under identical conditions and using standard assays for the determination of a dehydrogenase activity. The dehydrogenase activity can be monitored, for example, in a continuous spectrophotometric assay, when the enzyme, in pure form or in the form of a cell lysate, is incubated with substrate, i.e. 3-hydroxyisobutyric acid and redox factor and the reaction progress is monitored by means of the absorbance of the redox factor.

Different conditions are possible when carrying out the method according to the invention. The temperature may be more than 20° C., 30° C., 40, 50, 60, 70° C. or more than 80° C., preferably up to 100° C., provided that, in the case of the use of a living cell and of a suitable enzyme preparation, the selected cell and the selected enzymes are viable and show activity. It is known to those skilled in the art which organisms are viable at which temperatures, for example from text books such as Fuchs/Schlegel, 2007. In the case of a living yeast cell, the temperature can be 5 to 45° C., preferably 15 to 42° C., more preferably 20 to 30° C. In the case of a Gram-negative bacterium, preferably a bacterium from the Enterobacteriaceae family, most preferably E. coli, the temperature can be 5 to 45° C., preferably 15 to 42° C., more preferably 20 to 30° C., most preferably 35 to 40° C.

For culturing the cell according to the invention, numerous culture media are possible, for example, in the case of using a yeast cell, YPD, YPN and YNB, which may be supplemented with amino acids, for example with 0.01 g/l tryptophan, or with glucose, for example at a concentration of 1% (w/v). In the case of the use of a bacterium from the Enterobacteriaceae family, preferably E. coli, culturing is possible in complete media such as LB medium or high cell density medium (HCD medium) consisting of NH₄SO₄ 1.76 g, K₂HPO₄ 19.08 g, KH₂PO₄ 12.5 g, yeast extract 6.66 g, Na₃ citrate 1.96 g, NH₄Fe citrate (1%) 17 ml, US3 trace element solution 5 ml, feed solution (glucose 50% w/v, MgSO₄[×7H₂O 0.5% w/v, NH₄Cl 2.2% w/v) 30 ml per litre.

In a preferred embodiment, cells used in the method according to the invention are used in another medium to the one used for steps a) to d) of the method. In a particularly preferred embodiment, the medium used for the culture is a complete medium and the medium used for steps a) to d) is a minimal medium. The method according to the invention, if it is conducted using viable cells, is conducted, after culture of the cells, preferably in transformation buffer containing per litre (NH₄)H₂PO₄ 8 g, NaCl 0.5 g, MgSO₄×7H₂O 0.48 g, US3 trace element solution 15 ml. 1 litre of the US 3 trace element solution is composed of HCl 37% 36.5 g, MnCl₂×4H₂O 1.91 g, ZnSO₄×7H₂O 1.87 g, Na EDTA×2H₂O 0.8 g, H₃BO₃ 0.3 g, Na₂MoO₄×2H₂O 0.25 g, CaCl₂×2H₂O 4.7 g, FeSO₄×7H₂O 17.8 g, CuCl₂×2H₂O 0.15 g and its pH is adjusted to 5.4.

The concentration of isobutyric acid at the start of the reaction, if this is provided as ready-to-use reactant at the beginning of the method, is 0.01 to 2, preferably 0.05 to 1, most preferably 0.1 to 0.2% (w/v). If 3-hydroxyisobutyric acid is prepared using glucose as the carbon source, which is metabolised to suitable precursors by a competent strain, its concentration in the medium at the start is 1% (w/v).

The steps a) to d) of the method according to the invention are preferably conducted at atmospheric pressure. When preparing isobutyric acid from isobutane, it can be advantageous to conduct the alkane hydroxylase reaction in the presence of relatively high pressures, in the presence of a gas mixture comprising, preferably comprising predominantly, isobutane. In a preferred embodiment, the pressure is more than 1.5, 2, 3 or 4 bar. In a further embodiment, the pressure is 0.5 to 4, preferably 1 to 3, most preferably 1 to 1.5 bar.

In a most preferred embodiment of the present invention, the object of the invention is achieved wherein a cell of the genus Yarrowia, preferably Yarrowia lipolytica, for which the activity of the hydroxyisobutyric acid dehydrogenase Yali0F02607g, or a variant thereof, is reduced by deletion compared to the activity of the corresponding wildtype cell, is brought into contact with isobutyric acid in aqueous solution.

The present invention is further illustrated by the following figures and non-limiting examples, from which further characteristics, embodiments, aspects and advantages of the present invention may be inferred.

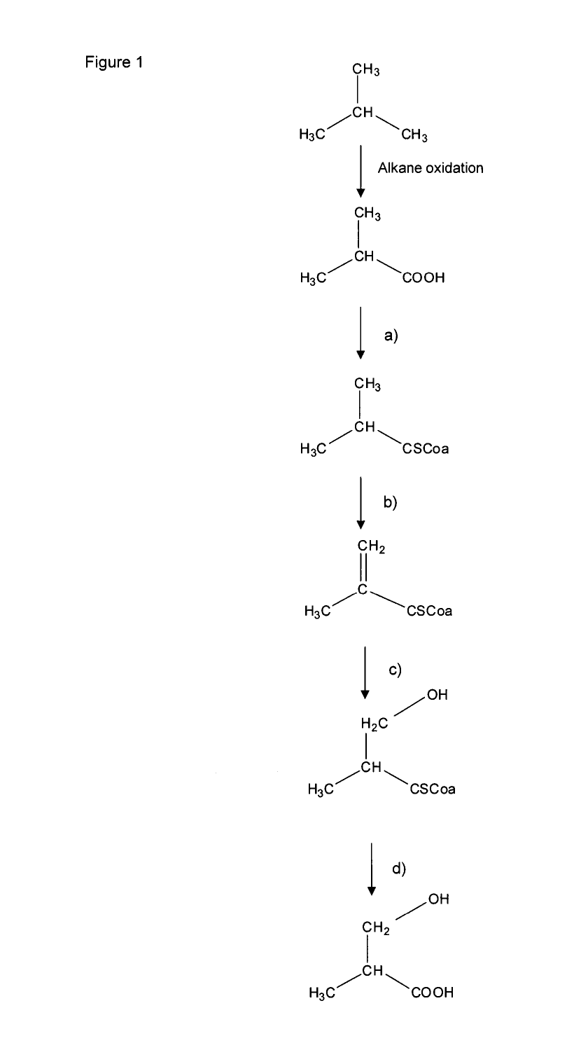

FIG. 1 shows a particularly preferred sequence according to the invention of enzymatically catalysed reactions comprising the conversion of isobutyric acid to isobutyryl-CoA by isobutyrate kinase and phosphotransisobutyrylase or isobutyryl-coenzyme A synthetase/ligase or isobutyrate-coenzyme A transferase in step b), the oxidation of isobutyryl-coenzyme A to methacrylyl-coenzyme A by isobutyryl-coenzyme A dehydrogenase in step c), the addition of water onto methacrylyl-coenzyme A with formation of 3-hydroisobutyryl-coenzyme A in step d) and its hydrolysis to 3-hydroxyisobutyric acid in step e).

### EXAMPLES

### Example 1

### Preparation of a Recombinant Yarrowia lipolytica Cell with Attenuated 3-Hydroxyisobutyric Acid Dehydrogenase Activity

### 1. Construction of the Gene Disruption Cassette GDC-Yl02607PT for the Deletion of the 3-Hydroxyisobutyric Acid Dehydrogenase Gene Yali0F02607g Accession Number: XP_{—}504911.1)

For the construction of a Yali0F02607g knockout mutant in Y. Lipolytica, the promotor and terminator region of the gene Yali0F02607g was cloned. Chromosomal DNA from Y. lipolytica H222 (MATa) served as matrix for the PCR. The gene knockout was conducted in the following strains: Y. lipolytica H222-41 (MATa ura3-41) and Y. lipolytica H222-SW4-2 (MATa ura3-302 SUC2 ku70Δ-1572 trp1Δ-1199). The promotor and terminator region of the gene coding for the 3-hydroxyisobutyric acid dehydrogenase (Yali0F02607g) was amplified in a PCR by means of the oligonucleotides 3HIBDH-Pfw (SEQ.-ID-No. 01), 3HIBDH-Pry (SEQ.-ID-No. 02) (promoter region), 3HIBDH-Tfw (SEQ.-ID-No. 03) and 3HIBDH-Try (SEQ.-ID-No. 04) (terminator region) from the chromosomal DNA of Y. lipolytica H222. The following parameters were used for the PCR: promoter region, 1×: initial denaturation, 98° C., 3 min; 35×: denaturation, 98° C., 0:10 min, annealing, 59.5° C., 0:45 min; elongation, 72° C., 0:35 min; 1×: terminal elongation, 72° C., 5 min. Terminator region, 1×: initial denaturation, 98° C., 3 min; 35×: denaturation, 98° C., 0:10 min, annealing, 59.5° C., 0:45 min; elongation, 72° C., 0:35 min; 1×: terminal elongation, 72° C., 5 min. For the amplification, the Phusion™ High-Fidelity Master Mix from New England Biolabs (Frankfurt) was used according to the manufacturer's recommendations. In this manner the promoter fragment was provided with an I-SceI restriction site at the 3′-end and the terminator fragment was provided with an I-SceI restriction site at the 5′-end. For this, the following oligonucleotides were used:

3HIBDH-Pfw:
(SEQ.-ID-No. 01)
5′- CAC ACA TCC AGA GCT CTA TG -3′
3HIBDH-Prv:
(SEQ.-ID-No. 02, containing an I-SceI
recognition sequence at the 5′-end)
5′-TAT ATA CTA TAT TAC CCT GTT ATC CCT AGC
GTA ACT ACA AAT ACA AGT TTT AAG CTG -3′
3HIBDH-Tfw:
(SEQ.-ID-No. 03, containing an I-SceI
recognition sequence at the 5′-end)
5′- TAT ATA AGT TAC GCT AGG GAT AAC AGG GTA
ATA TAG GCT GTG TAT GTG TTA GGG TG -3′
3HIBDH-Trv:
(SEQ.-ID-No. 04)
5′- GGT GAC CTT CAG GTG CAC CA -3′

### 2. Fusion of the Promoter and Terminator Fragment

The PCR products of the promotor and terminator region (1060 and 970 base pairs respectively) were purified using the “QIAquick PCR-Purification Kits” (Qiagen, Hilden) according to the manufacturer's instructions. In a subsequent crossover PCR, the two PCR products were used as matrix and an amplification was conducted with the primers 3HIBDH-Pfw (SEQ.-ID-No. 01) and 3HIBDH-Trv (SEQ.-ID-No. 04). The following parameters were used for the PCR: 1×: initial denaturation, 98° C., 3 min; 35×: denaturation, 98° C., 0:10 min, annealing, 59.5° C., 0:45 min; elongation, 72° C., 1:00 min; 1×: terminal elongation, 72° C., 5 min. A 2.071 kilobase pair PCR product (SEQ.-ID-Nr. 05) was formed by the complementarity of the I-SceI restriction site. To isolate the DNA from an agarose gel the target DNA was excised from the gel with a scalpel and purified with the “Quick Gel Extraction Kit” (Qiagen, Hilden). This was conducted according to the manufacturer's instructions. In the next step the PCR product was ligated into the pCR-Blunt II-Topo (Zero Blunt TOPO PCR Cloning Kit with One Shot TOP10 Chemically Competent E. coli, Invitrogen, Karlsruhe) vector. The resulting pCRBluntIITopo::P_T_{—}3 HIBDH_Yl plasmid (SEQ.-ID-No. 06) is 5.59 kilobase pairs long. The ligation and the chemically competent E. Coli transformation were carried out according to the manufacturer's instructions. The authenticity of the plasmid was checked by a restriction analysis with EcoRI, XhoI and PstI.

3. Construction of the Knockout Mutants Y. lipolytica H222-SW-4-2 Δ 3HIBDH and Y. lipolytica H222-41 Δ3HIBDH

For construction of the knockout mutant, the integration of a marker gene is required. For this purpose, the 1.3 kilobase pair “loxP-URA3-loxR” DNA fragment, which contains the URA3 gene, was excised from the pJMP113 (Fickers et al., 2003) vector by means of a I-SceI restriction. The “loxP-URA3-loxR” fragment was purified over on an agarose gel using a “Quick Gel Extraction Kit” (Qiagen, Hilden) according to the manufacturer's instructions. The resulting fragment was ligated into the pCRBluntIITopo::P_T_{—}3 HIBDH_Yl vector (SEQ.-ID-No. 06), cleared with I-SceI. The resulting plasmid pCRBluntIITopo::P_T_{—}3 HIBDH_Yl_ura (SEQ.-ID-No. 07) is 6.899 kilobase pairs in size. The ligation and also the transformation of chemically competent E. coli DH5α cells (New England Biolabs, Frankfurt) was carried out in a manner known to those skilled in the art. The authenticity of the plasmid was checked by a restriction with XmaI, ScaI and SacI.

In order to obtain the gene disruption cassette GDC-Yl02607PT for the deletion of ORFYaliOF02607g required for the gene knockout, the plasmid pCRBluntIITopo::P_T_{—}3 HIBDH_Yl_ura (SEQ.-ID-No. 07) was used as matrix for the PCR with the following oligonucleotides and parameters: 3HIBDH-Pfw (SEQ.-ID-No. 01) and 3HIBDH-Try (SEQ.-ID-No. 04); 1×: initial denaturation, 98° C., 3 min; 35×: denaturation, 98° C., 0:10 min, annealing, 65° C., 0:45 min; elongation, 72° C., 1:45 min; 1×: terminal elongation, 72° C., 5 min. The desired 3.38 kilobase pair fragment (SEQ.-ID-No. 8) was purified by means of a “Quick Gel Extraction Kit” (Qiagen, Hilden) on an agarose gel, according to the manufacturer's instructions, checked by the restriction with XmaI and XmnI and used for the integrative transformation of Y. lipolytica H222-SW4-2 and Y. lipolytica H222-41.

The transformation was conducted using the lithium acetate method (Barth G and Gaillardin C (1996) Yarrowia lipolytica. In: Wolf, K. (eds) Nonconventional yeasts in biotechnology. Springer, Berlin Heidelberg N.Y., pp 313-388). The uracil prototrophic transformants obtained were checked by colony PCR. The following parameters were used for the PCR: 1×: initial denaturation, 98° C., 3 min; 35×: denaturation, 98° C., 0:10 min, annealing, 60° C., 0:45 min; elongation, 72° C., 1:30 min; 1×: terminal elongation, 72° C., 5 min. For this, the following oligonucleotides were used:

fw-3HIBDH-ah:
(SEQ.-ID-No. 09)
5′- GAG TCG CAG ATT CAG GAA AT -3′
rv-3HIBDH-ah:
(SEQ.-ID-No. 10)
5′- TCA CCT TCT GAT CAC GGT GT -3′

In the case of a successful disruption of the Yali0F02607g gene coding for the 3-hydroxyisobutyric acid dehydrogenase, a 1.005 kilobase pair fragment should be amplified. In fact, corresponding clones could be identified, which were further processed below.

### 4. Restoration of the Uracil Auxotrophy

The restoration of the uracil auxotrophy in the uracil prototrophic transformants was carried out according to Fickers et al., 2003. Initially, competent cells were produced. For this, the transformants were cultured in 5 ml of YPD pH 4 (10 g/l yeast extract, 10 g/l peptone, 10.5 g/l citric acid, 2% (w/v) glucose and 0.5 M sodium citrate to adjust the pH) for 8 h at 30° C. and 190 rpm in 100 ml baffled flasks. After 8 h this pre-culture was used to inoculate 10 ml of YPD medium pH 4 in 250 ml baffled flasks with an optical density (OD600) of 0.05. All flasks were incubated at 30° C. and 190 rpm. On the following day these pre-cultures were harvested (500 g, 5 min, RT) on reaching a cell count between 9·107 and 1.5·107 per ml (counting using a Neubauer chamber), washed twice in 10 ml of TE buffer (10 mM Tris-HCl, 1 mM Na EDTA, pH 8), subsequently diluted in 30 ml of 0.1% (w/v) lithium acetate and incubated for 1 h at 28° C. and 60 rpm. Subsequently the cells were again centrifuged (500 g, 5 min, RT), resuspended in 3 mL of 0.1% (w/v) lithium acetate, aliquoted in 100 μl portions and used immediately. All further steps were conducted on ice.

For the transformation of the pUB4Cre plasmid (Fickers et al., 2003), 100 μl of the competent cells were mixed with 200-800 ng plasmid DNA and 2.5 μL of boiled salmon sperm DNA (10 mg/ml) (Invitrogen, Karlsruhe). After addition of 0.7 mL of 40% PEG4000 (w/v; dissolved in 0.1 M lithium acetate, pH 6) the cells were incubated for 1 h at 28° C. with shaking. Heat shock at 39° C. followed for 10 min in a water bath. To the cells were added 1.2 mL of 0.1% (w/v) lithium acetate and 2×250 μl were very carefully applied to YPD plates with 500 μg/ml hygromycin.

After incubation for 3 to 10 days at 30° C., the clones obtained were checked phenotypically on agar plates with YNB medium (6.7 g/l Difco™ yeast nitrogen base w/o amino acids) and glucose with or without uracil. In order to remove the pUB4Cre plasmid from the cell, the transformants were incubated in 10 ml of YPD medium in several 100 ml baffled flasks at 30° C. and 190 rpm for 24 h and streaked on YPD plates. The resulting clones were checked phenotypically on agar plates and by PCR (as described in section 3).

In this manner the following strains were constructed:
H222-SW4-2 Δ3HIBDH
H222-41□ Δ3HIBDH

### Example 2

### Production of 3-Hydroxyisobutyric Acid with Glucose as the Sole Carbon Source by Means of Genetically Modified Y. lipolytica Cells, in which the 3-Hydroxyisobutyric Acid Dehydrogenase Activity has been Attenuated

The Y. lipolytica H222-41 Δ3HIBDH strain constructed in example 1, alongside the corresponding wildtype H222-41, were cultured in 10 mL of YNB medium (6.7 g/l Difco™ yeast nitrogen base w/o amino acids) containing 0.2 g/l uracil, 0.01 g/l tryptophan and 5% (w/v) glucose overnight at 28° C. and 190 rpm. The following morning, these pre-cultures were each used to inoculate 20 mL of YNB medium containing 0.2 g/l uracil, 0.01 g/l tryptophan and 1% (w/v) glucose in 100 ml baffled flasks with an optical density (OD600) of 0.5. All flasks were incubated at 28° C. and 190 rpm.

After a time period of 70 h, 1 g/l of ammonium sulphate was added and after 95 h the concentrations of 3-hydroxyisobutyric acid in the mixtures were analysed by IC. For the chromatographic separation in the ICS-2000 RFIC (Dionex, Corporation, Sunnyvale, USA) a RFIC™ TonPac column (2×250 mm, column temperature 30° C., +precolumn AG15 4×50 mm, flow rate 0.38 ml/min) was used.

The strains reached an OD600 of ca. 30. Whereas the control strain Y. lipolytica H222-41 did not produce any 3-hydroxyisobutyric acid, in the case of the Y. lipolytica H222-41 Δ3HIBDH derived from it, the formation of 4.5 mg/l 3-hydroxyisobutyric acid could be detected.

### Example 3

### Production of 3-Hydroxyisobutyric Acid Starting from Isobutyric Acid with Genetically Modified Y. lipolytica Cells, in which the 3-Hydroxyisobutyric Acid Dehydrogenase Activity has been Attenuated

The Y. lipolytica strains H222-SW-4-2 Δ3HIBDH and H222-41 Δ3HIBDH constructed in example 1 were cultivated, alongside an unmodified control strain (H222-SW-4-2), in 10 mL of YNB medium (6.7 g/l Difco™ yeast nitrogen base w/o amino acids) containing 0.01 g/l tryptophan and 1% (w/v) glucose at 30° C. and 190 rpm overnight. The following morning these precultures were each used to inoculate 20 mL of YNB medium containing 0.2 g/l uracil, 0.01 g/l tryptophan, 1% (w/v) glucose and 0.2% (w/v) isobutyric acid (titrated with NaOH, pH: 5.1; after 24 h 0.2% isobutyric acid was freshly added) in 100 ml baffled flasks with an optical density (OD600) of 0.5. All flasks were incubated at 30° C. and 190 rpm. The H222-SW4-2-strains reached an OD600 of 2-3 whereas the H222-41 strains grew to an OD600 of ca. 10.

At 24 h and 48 h time points the concentrations of 3-hydroxyisobutyric acid in all mixtures were analysed by IC. (see FIG. 1). The culture supernatant was diluted with ddH₂O 1:10, such that the measured values were in the calibration range. For the chromatographic separation in the ICS-2000 RFIC (Dionex, Corporation, Sunnyvale, USA) a RFIC™ TonPac column (2×250 mm, column temperature 30° C., +precolumn AG15 4×50 mm, flow rate 0.38 ml/min) was used.

After 24 h all the Y. lipolytica strains reached the stationary growth phase. The pH for the H222-41 strains declined during the growth to 3.3 to 3.7, while a pH of 2 was detected for the H222-SW-4-2 strains. After 24 h and 48 h the modified Y. lipolytica strains H222-SW-4-2 Δ3HIBDH and H222-41 Δ3HIBDH Δ3HIBDH and H222-41 Δ3HIBDH reached a higher yield than the respective unmodified strains H222-SW-4-2 and H222-41. Y. lipolytica H222-41Δ3HIBDH converts over 90% isobutyric acid to 3-hydroxyisobutyric acid after 24 h and over 80% after 48 h.

By deletion of Yali02607g in Y. lipolytica H222-41 and H222-SW-4-2 the yield of 3-hydroxyisobutyric acid production from isobutyric acid could be considerably increased (see FIG. 1).

### Example 4

### Production of 3-Hydroxybutyric Acid (3-HIB) from Glucose or Isobutyric Acid (IBA) as the Sole Carbon Source by Y. lipolytica H222 (Wildtype, WT) and Genetically Modified Y. lipolytica H222 Δ3HIBDH (ura)-8 with Attenuated 3-Hydroxyisobutyric Acid Dehydrogenase Activity (Δ)

a) Biomass Production on a Shaking Flask Scale
Cryocultures of Y. lipolytica H222 Δ3HIBDH (ura)-8 (in the following denoted as A) and Y. lipolytica H222 (in the following denoted as wildtype (WT)) were autoclaved on YPD agar plates per litre prepared from yeast extract 10 g, peptone 20 g, agar agar 12 g, and complemented with separately autoclaved glucose 10 g, plated out and incubated for 24 h at 28° C.
Per strain, 2 1000 ml shake flasks with baffles were charged with 100 ml of YPD medium (above medium without agar agar, each with 3 drops of Delamex antifoam), inoculated in each case with 2 full inoculation loops from the agar plates and incubated for 20 h at 30° C. and 180 rpm (amplitude 2.5 cm) (residual glucose 0 g/l, OD≧20).

TABLE 1

| Strain | pH | OD | Glucose [g/l] |
|---|---|---|---|
| Δ | 7.06 | 20.9 | 0.029 |
| WT | 7.47 | 23.7 | 0.015 |

The cultures were then placed under sterile conditions in 50 ml falcon tubes and centrifuged at 5000 rpm. The pellets were washed 4× with 0.9% saline. Then, 2 pellets of each strain were resuspended in 50 ml of transformation buffer and combined.
The transformation buffer was composed per sterile-filtered litre of (NH₄)H₂PO₄ 8 g, NaCl 0.5 g, MgSO₄×7H₂O 0.48 g, trace element solution US3 15 ml. 1 litre of the US3 trace element solution US 3 was composed of HCl 37% 36.5 g, MnCl₂×4H₂O 1.91 g, ZnSO₄×7H₂O 1.87 g, Na EDTA×2H₂O 0.8 g, H₃BO₃ 0.3 g, Na₂MoO₄×2H₂O 0.25 g, CaCl₂×2H₂O 4.7 g, FeSO₄×7H₂O 17.8 g, CuCl₂×2H₂O 0.15 g. This solution was separately sterile-filtered and added to the buffer under sterile conditions. The pH of the buffer was adjusted to 5.4.

b) Biotransformation
To each of 4 1000 ml shaking flasks with baffles were added, under sterile conditions, 50 ml of the said transformation buffer pH 5.4, each with 3 drops of Delamex antifoam. For each strain, 0.2% (w/v) isobutryic acid was added, under sterile conditions, to one shaking flask to a final volume of 100 ml, and 1% (w/v) glucose was added to a second shaking flask. One shaking flask with IBA and one with glucose were each inoculated with 50 ml of resuspended pellet of Y. lipolytica H222 from a). The Y. lipolytica H222 Δ3HIBDH (ura)-8 was likewise processed. The initial OD was ca. 14. The shaking flasks were incubated at 30° C. and 180 rpm. The sampling was carried out after 0.6 and 24 hours. Microscopic controls revealed no lysed cells in any culture over the entire time. The glucose was measured with a YSI measuring device supplied by the Kreinbaum company, the isobutyric acid and 3-hydroxyisobutyric acid was measured by HPLC using an Aminex column and the OD was measured with a Spectralphotometer at 600 nm.

Results:

TABLE 2
OD

| t [h] | WT IBA | WT Glc | Δ IBA | Δ Glc |
|---|---|---|---|---|
| 0 | 13.8 | 13.6 | 15.0 | 13.6 |
| 6 | 13.5 | 19.2 | 12.8 | 25.3 |
| 24 | 13.3 | 18.2 | 10.6 | 16.4 |

The glucose was partly metabolised to the structure of biomass. With IBA as the sole carbon source, no biomass production took place.

TABLE 3
Substrate concentration of IBA/Glucose [mg/l]

| Strain | WT IBA | | WT Glucose | | Δ IBA | | Δ Glucose | |
|---|---|---|---|---|---|---|---|---|
| t [h] | IBA | Glucose | IBA | Glucose | IBA | Glucose | IBA | Glucose |
| 0 | 1914 | 20 | 0 | 8450 | 1808 | 10 | 0 | 8520 |
| 6 | 676 | 0 | 0 | 3240 | 1267 | 0 | 0 | 1770 |
| 24 | 0 | 0 | 0 | 0 | 422 | 0 | 0 | 0 |

Both substrates were metabolised both by Y. lipolytica H222, and by Y. lipolytica H222 Δ3HIBDH (ura)-8.

TABLE 4
Concentration of 3-HIB [mg/l]

| t [h] | WT IBA | WT Glucose | Δ IBA | Δ Glucose |
|---|---|---|---|---|
| 0 | 1 | 1 | 10 | 5 |
| 6 | 50 | 0 | 821 | 21 |
| 24 | 0 | 0 | 2042 | 52 |

In the case of Y. lipolytica H222, only with IBA could 3-HIB be briefly detected as a metabolic product.
With Y. lipolytica H222 Δ3HIBDH (ura)-8 and glucose as substrate, low amounts of free 3-HIB could be detected. Of the IBA used, 15.7 mmol/l were consumed. From this, 18.6 mmol/l can theoretically be formed. 19.6 mmol/l were measured. The IBA consumed is therefore completely converted to 3-HIB.

### Example 5

### Production of 3-Hydroxyisobutyric Acid Starting from Ketoisovalerate with Genetically Modified Y. Lipolytica Cells, in which the 3-Hydroxyisobutyric Acid Dehydrogenase Activity has been Attenuated

The Y. lipolytica strain H222-41 Δ3HIBDH constructed in example 1, alongside the corresponding wildtype Y. lipolytica H222-41, was cultured in 10 mL of YNB medium (6.7 g/l Difco™ yeast nitrogen base w/o amino acids) containing 0.2 g/l uracil, 0.01 g/l tryptophan and 1% (w/v) glucose overnight at 30° C. and 190 rpm. On the following day, these pre-cultures were each used to inoculate 25 mL of YNB medium containing 0.2 g/l uracil, 0.01 g/l tryptophan, 6% (w/v) glucose and 0.65% (w/v) ketoisovalerate (after 25.5 h 0.5% (w/v) ketoisovalerate was freshly added to the culture and 0.3% (w/v) ketoisovalerate was added after 49.5 h) in 100 ml baffle flasks with an optical density (OD600) of 0.5. All flasks were incubated at 30° C. and 190 rpm.

Over the course of time, the optical density (OD600) was determined and the 3-hydroxyisobutyric acid content was analysed by IC in all mixtures. During the culture period, sufficient glucose and ketoisovalerate was at all times present in the medium. After ca. 24 h, all Y. lipolytica strains reached the stationary growth phase. During the growth, the pH dropped to ca. 3.

By deletion of Yali0F02607g in Y. lipolytica H222-41 the 3-hydroxyisobutyric acid production from ketoisovalerate could be increased from 2 g/l of 3-hydroxyisobutyric acid to more than 5 g/l of 3-hydroxyisobutyric acid (see FIG. 2).

### Example 6

### Production of 3-Hydroxyisobutyric Acid by a 2 Step Process, Starting from Isobutane, Using a Genetically Modified E. coli W3110 with the Monooxygenase (alkBGT) from P. putida GPO1, to Give Isobutyric Acid, which is Further Converted by Genetically Modified Y. lipolytica H222 (Ura)-8, in which the 3-Hydroxyisobutyric Acid Dehydrogenase Activity has been Attenuated, with IBA as the Sole Carbon Source

### Step 1

Production of isobutyric acid from isobutane by E. coli W3110 with the monooxygenase (alkBGT) from P. putida GPO1.

a) Production of Biomass on a 10 l Scale
Pre-seeding culture: 1 litre of LB Medium with 50 μl of kanamycin was prepared from a solution of yeast extract 5 g, peptone 10 g, NaCl 0.5 g and 50 μl of kanamycin. The pH was adjusted to 7.4 with 5% NH₄OH. The solution is autoclaved for 20 minutes at 121° C.
5×25 ml of this solution were placed in 100 ml shake flasks with baffles and each was inoculated with 200 μl of a glycerol cryoculture of E. coli W3110 pBT10 (DE10200710060705). These cultures were incubated for 18 hours at 37° C. and 180 rpm (amplitude 2.5 cm).
Seeding culture: 1 litre of high cell density medium (HCD medium) consisting of NH₄SO₄ 1.76 g, K₂HPO₄ 19.08 g, KH₂PO₄ 12.5 g, yeast extract 6.66 g, Na₃ citrate 1.96 g, NH₄FE citrate (1%) 17 ml, US3 trace element solution 5 ml, feed solution (glucose 50% w/v, MgSO₄×7H₂O 0.5% w/v, NH₄Cl 2.2% w/v) 30 ml, and also 50 μg kanamycin was made up with demineralised water. 1 litre of the US 3 trace element solution comprised HCl 37% 36.5 g, MnCl₂×4H₂O 1.91 g, ZnSO₄×7H₂O 1.87 g, Na EDTA×2H₂O 0.8 g, H₃BO₃ 0.3 g, Na₂MoO₄×2H₂O 0.25 g, CaCl₂×2H₂O 4.7 g, FeSO₄×7H₂O 17.8 g, CuCl₂×2H₂O 0.15 g. 948 ml of solution with NH₄SO₄ to Na₃-citrate are autoclaved, the remainder were in each case separately sterile filtered and subsequently added under sterile conditions. The pH was 6.8.
5×75 ml of the HCD medium in 1000 ml shake flasks with baffles were each inoculated with 25 ml of pre-seed culture and cultured for 30 h at 37° C. and 180 rpm (amplitude 2.5 cm). Induction culture: Each 25 ml of the culture broth were used as inoculum for 75 ml of modified M9 medium (sterile filtered) with the following composition per litre: 15 g glucose, 6.79 g Na₂PO4, 3 g KH₂PO₄, 0.5 g NaCl, 2 g NH₄Cl, 15 g yeast extract, 0.49 g MgSO₄*7H₂O, 1 ml trace element solution (as in the seed culture) and 50 μg kanamycin in 1000 ml shake flasks. The cultures are incubated for 7 h at 35° C. and 180 rpm (amplitude 2.5 cm).
A sterile 10 l fermenter was charged with 7 I of a sterile medium with the composition (per litre) (NH₄)₂SO₄ 1.75 g, K₂HPO₄×3 H₂O 19 g, KH₂PO₄ 12.5 g, yeast extract 6.6 g, Na₃ citrate×2H₂O 2.24 g, glucose 15 g, MgSO₄×7H₂O 0.49 g, NH₄Fe citrate (1% w/v) 16.6 ml, trace element solution (as in the seed culture) 15 ml and kanamycin 50 μg and 2 ml of Delamex antifoam. As feed, an autoclaved solution of glucose (50% w/v) with MgSO₄×7H₂O 10 g/l was connected, for the purposes of pH correction with 0.5M H₂SO₄ and 25% NH₄OH.

The cultures from the shaking flasks were combined under sterile conditions and, by means of a transfer bottle, inoculated into the fermenter. The fermentation conditions were adjusted to: pO₂ 30%, airflow 6 nlpm, stirrer 400-1200 rpm, temperature 37° C., pH7, feed start 8 h, feed rate 150-250 g/h. After 19 h the temperature was lowered to 30° C., the feed was stopped and the culture induced with 0.4 mM DCPK. After 23 hours the OD in the fermenter was ca. 100, the culture broth was removed under sterile conditions and centrifuged at 8000 rpm with 500 ml in 1000 ml centrifuge tubes. The supernatant was discarded and the pellets were aliquoted in sterile Falcon tubes such that a resuspended pellet in 180 ml of transformation buffer gives an OD of ca. 20. The pellets could then be used immediately in the biotransformation, or be frozen at −80° C. for later use.

b) Biotransformation of Isobutane to Isobutyric Acid
The pellets from 200 ml of culture were resuspended in 10 ml of conversion buffer. The conversion buffer consisted of 70 mM (NH₄)H₂PO₄ buffer, pH 7 containing per litre 8 g (NH₄)H₂PO₄, 0.5 g NaCl, 0.49 g MgSO₄×7H₂O, 1 ml TE and 50 μg kanamycin. The pH adjustment here was carried out with 5% NH₄OH.
150 ml of buffer with ca. 3 drops of autoclaved antifoam (Delamex) were placed in a 300 ml fermenter. The fermenter was flushed with a gas mixture from a gas cylinder to an initial pressure of 5 bar with 25% isobutane and 75% synthetic air by means of a metal sinter perlator with a pore size of 0.2 μm with a flow rate of 12.5 l/h (stp). The fermenter was maintained at 35° C. in a water bath and stirred at 900 rpm by means of a magnetic stirrer. The waste air was discharged through a wash bottle filled with 150 ml water.
The fermenter was inoculated via a sampling tube with 30 ml of a pellet produced in a) and resuspended in buffer. The reaction was initiated by the start of a glucose feed (or in the case of iso also glucose batch or by initial charge+feed . . . ) of 1.5 g/lh. After 4.5 hours, a concentration of isobutyric acid of >350 mg/l was reached (3.97 mmol/kg).
The pH was adjusted to 5.4 with 1M H₂SO₄ and the gassing was readjusted to compressed air with a flow rate of 12.5 l/h (stp), and the temperature lowered to 30 degrees. (alternatively):
   From each of the fermenters 50 ml of transfer buffer were removed under sterile conditions by canulae and syringes for resuspending the Yarrowia cultures (see step 2a)
   the culture was harvested and the biomass was separated off by centrifugation at 8000 rpm with 500 ml in 1000 ml centrifuge tubes, and the supernatant was stored at −20° C. until required for transformation with the Yarrowia cultures.

### Step 2

Production of 3-Hydroxyisobutyric Acid from Isobutyric Acid from Step 1 by Genetically Modified Y. lipolytica H222 Δ3HIBDH (ura)-8 compared to the wildtype Y. lipolytica H222.

a) Production of Biomass on a Shake Flask Scale (or Alternatively Also in the Fermenter)
Cryocultures of Y. lipolytica H222 Δ3HIBDH (ura)-8 and Y. lipolytica H222 were autoclaved on YPD agar plates per litre prepared from yeast extract 10 g, peptone 20 g, agar agar 12 g, and complemented with separately autoclaved glucose 10 g, plated out and incubated for 24 h at 28° C.
Per strain, 2 1000 ml shake flasks with baffles were charged with 100 ml of YPD medium (above medium without agar agar, each with 3 drops of Delamex antifoam), inoculated in each case with 2 full inoculation loops from the agar plates and incubated for 20 h at 30° C. and 180 rpm (amplitude 2.5 cm) (residual glucose 0 g/l, OD≧20).
The cultures were then discharged under sterile conditions into 50 ml falcon tubes and centrifuged at 5000 rpm. The pellets were washed 4× with 0.9% saline. Then, 2 pellets of each strain were resuspended in 50 ml of transformation buffer—fresh or thawed (from step 1 b) and combined.

b) Biotransformation of Isobutyric Acid to 3-Hydroxyisobutyric Acid
Each of the 50 ml of the resuspended pellets of Y. lipolytica H222 Δ3HIBDH (ura)-8 and Y. lipolytica H222 were inoculated in a 300 ml fermenter with the transformation buffer from step 1 b) and stirred for 24 h at pH 5.4, 30° C. and 900 rpm and gassed with 1.2 vvm compressed air.
After 24 hours, no isobutyric acid and no 3-hydroxyisobutyric acid could be detected in the supernatant for the wildtype Y. lipolytica H222. After 24 h, in the case of Y. lipolytica H222 Δ3HIBDH (ura)-8, isobutyric acid could no longer be detected in the supernatant. With regard to 3-hydroxyisobutyric acid, concentrations were of >413 mg/l, corresponding to the isobutyric acid values of >3.97 mmol measured at the end of step 1 b).

### BIBLIOGRAPHY

A. Cornish-Bowden (1995), Fundamentals of Enzyme Kinetics, Portland Press Limited, 1995
DE 60216245 (2007): FUNCTIONAL DISPLAY OF POLYPEPTIDES
DE 10200710060705 (2007): ω-Aminocarbonsäuren oder ihre Lactame, herstellende, rekombinante Zellen (Recombinant cells producing w-aminocarboxylic acids or their lactams).
Sambrook/Fritsch/Maniatis (1989): Molecular Cloning: A Laboratory Manual, Cold Spring Harbor Laboratory Press, 2^{nd} edition
William Bauer, Jr. “Methacrylic Acid and Derivatives” in Ullmann's Encyclopedia of Industrial Chemistry 2002, Wiley-VCH, Weinheim
Hasegawa et al. (1981), J. Ferment. Technol. 59, pp 203-208
Hasegawa (1981b), Agric. Biol. Chem. 45, pp 2899-2901.
Hasegawa et al. (1982), J. Ferment. Technol. 60, pp 501-508
WO 2007/141208: MICROBIOLOGICAL PRODUCTION OF 3-HYDROXYISOBUTYRIC ACID
WO 2008/119738: ENZYME FOR THE PRODUCTION OF METHYLMALONYL COENZYME A OR ETHYLMALONYL COENZYME A, AND USE THEREOF
Patel R N et al. (1983), Journal of Applied Biochemistry 5 (1-2), 107-120
Barth G and Gaillardin C (1996) Yarrowia lipolytica. In: Wolf, K. (eds) Nonconventional yeasts in biotechnology. Springer, Berlin Heidelberg N.Y., pp 313-388
Van Beilen et al. (2002): Functional Analysis of Alkane Hydroxylases from Gram-Negative and Gram-Positive Bacteria”, Journal of Bacteriology, 2002, 184 (6), pp. 1733-1742).
Fersht A R and Winter G (2008): Redesigning Enzymes by Site-Directed Mutagenesis, Ciba Foundation Symposium 111—Enzymes in Organic Synthesis
Fuchs/Schlegel (2007) Allgemeine Mikrobiologie (General Microbiology), 2008, Georg Thieme Verlag
William Bauer, Jr. “Methacrylic Acid and Derivatives” in Ullmann's Encyclopedia of Industrial Chemistry 2002, Wiley-VCH, Weinheim

1. A method, comprising:
a) contacting isobutyric acid with
a combination of isobutyrate kinase and phosphotransisobutyrylase,
isobutyryl-coenzyme A synthetase/ligase,
isobutyrate-coenzyme A transferase, or any mixture thereof to obtain a first intermediate product;
b) contacting the first intermediate product with isobutyryl-coenzyme A dehydrogenase to obtain a second intermediate product;
c) contacting the second intermediate product with methacrylyl-coenzyme A hydratase to obtain a third intermediate product; and
d) hydrolyzing the third intermediate product to form 3-hydroxyisobutyric acid,
wherein at least one of the enzymes used in the contacting a), b) and c) from the group comprising isobutyrate kinase, phosphotransisobutyrylase, isobutyryl-coenzyme A synthetase/ligase and isobutyrate-coenzyme A transferase, is in the form of a cell, which, compared to its wildtype, has a reduced activity of a 3-hydroxyisobutyric acid dehydrogenase or a variant thereof.

2. The method according to claim 1, wherein the isobutyric acid is formed by bringing isobutane into contact with a monooxygenase.

3. The method according to claim 1, wherein the hydrolysis in the contacting c) is achieved by bringing the second intermediate product from c) into contact with a 3-hydroxyisobutyryl-coenzyme A hydrolase.

4. The method according to claim 1, wherein the cell comprises both the isobutyryl-coenzyme A dehydrogenase in the contacting b), the methacrylyl-coenzyme A hydratase in the contacting c) and
the combination of isobutyrate kinase and phosphotransisobutyrylase,
isobutyryl-coenzyme A synthetase/ligase,
isobutyrate-coenzyme A transferase, or any mixture thereof.

5. The method according to claim 1, wherein the cell further comprises an alkane hydroxylase.

6. The method according to claim 1, wherein the 3-hydroxyisobutyric acid dehydrogenase is XP_{—}504911.1 or a variant thereof.

7. A cell, comprising at least one enzyme selected from a group comprising isobutyryl-coenzyme A synthetase/ligase, isobutyrate-coenzyme A transferase, isobutyrate kinase, phosphotransisobutyrylase, isobutyryl-coenzyme A dehydrogenase and methacrylyl-coenzyme A hydratase and, has compared to its wildtype, a reduced activity of a 3-hydroxyisobutyric acid dehydrogenase or a variant thereof.

8. The cell according to claim 7, wherein the cell further comprises, in addition to an isobutyryl-coenzyme A dehydrogenase, and in addition to a methacrylyl-coenzyme A hydratase,
a combination of isobutyrate kinase and phosphotransisobutyrylase,
isobutyryl-coenzyme A synthetase/ligase,
isobutyrate-coenzyme A transferase or any mixture thereof.

9. The cell according to claim 7, further comprising an alkane hydroxylase.

10. The cell according to claim 6, wherein the 3-hydroxyisobutyric acid dehydrogenase is XP_{—}504911.1 or a variant thereof.

11. The cell according to claim 7, wherein the cell is suitable for preparing 3-hydroxyisobutyric acid.

12. The cell according to claim 11, wherein the 3-hydroxyisobutyric acid dehydrogenase is XP_{—}504911.1 or a variant thereof.

13. The method according to claim 1, wherein the cell is a bacterial or lower eukaryotic cell.

14. The method according to claim 1, wherein the cell comprises a yeast cell from the group of genera which comprises Yarrowia, Candida, Saccharomyces, Schizosaccharomyces and Pichia.

15. A reaction mixture, comprising the cell according to claim 7, and also isobutane or isobutyric acid.

16. The method according to claim 1, wherein the isobutyric acid is formed by bringing isobutane into contact with an alkane hydroxylase.

17. The method according to claim 1, wherein the isobutyric acid is formed by bringing isobutane into contact with an alkane hydroxylase which is one of the alk-BGT type or a variant thereof.

18. The cell according to claim 7, further comprising an alkane hydroxylase which is one of the alkBGT type or a variant thereof.

19. The method according to claim 1, wherein the cell comprises a yeast cell comprising Yarrowia lipolytica.

20. The method according to claim 1, wherein all of the enzymes used in the contacting a), b) and c) from the group comprising isobutyrate kinase, phosphotransisobutyrylase, isobutyryl-coenzyme A synthetase/ligase and isobutyrate-coenzyme A transferase, is in the form of a cell, which, compared to its wildtype, has a reduced activity of a 3-hydroxyisobutyric acid dehydrogenase or a variant thereof.
